(19) United States
(12) Patent Application Publication
(10) Pub. No.: US 2012/0003657 A1
(43) Pub. Date: Jan. 5, 2012

(54) TARGETED SEQUENCING LIBRARY PREPARATION BY GENOMIC DNA CIRCULARIZATION

(76) Inventors: Samuel Myllykangas, Espoo (FI); Hanlee P. Ji, Stanford, CA (US)

(21) Appl. No.: 13/174,297

(22) Filed: Jun. 30, 2011

Related U.S. Application Data
(60) Provisional application No. 61/398,886, filed on Jul. 2, 2010.

(51) Int. Cl. C12Q 1/68

(52) U.S. Cl. 435/6.12; 435/6.1

(57) ABSTRACT

Certain embodiments provide a method of sequencing that comprises: a) contacting, under hybridization conditions, a target genomic fragment with: i. a vector oligonucleotide comprising a binding site for a sequencing primer; and ii. a splint oligonucleotide that hybridizes to the vector oligonucleotide and to the nucleotide sequences at the ends of a target genomic fragment, to produce a circular nucleic acid; b) contacting the circular nucleic acid with a ligase, thereby ligating the ends of the vector oligonucleotide to the ends of the target genomic fragment to produce a circular DNA molecule; c) separating the circular DNA molecule from the splint oligonucleotide; and d) sequencing the target genomic fragment of the circular DNA molecule using the first sequencing primer.

### CROSS-REFERENCING

This application claims the benefit of U.S. provisional application Ser. No. 61/398,886, filed on Jul. 2, 2010, which application is incorporated by reference herein in its entirety.

### GOVERNMENT RIGHTS

This work was made with Government support under contract 2P01HG000205 awarded by the National Institutes of Health. The Government has certain rights in this invention.

### BACKGROUND

The wave of new technologies and biochemistry that have enabled mass parallelization and high-throughput imaging of cyclic sequencing reactions on solid surface has substantially increased the ability to accumulate genetic information. The “next-generation sequencing” technologies provide powerful tools for understanding diseases like cancer that are predominantly defined by genetic, genomic and epigenetic alterations in the somatic or germline cells. For example, cancer is a heterogeneous group of diseases originating from different tissues and presented with a complex repertoire of genetic alterations.

Typically, preparation of samples for next-generation sequencing involves complicated molecular biology processes that ensure that specific adaptor sequences are added to the ends of the analyzed genomic DNA fragments. This preparation of recombinant DNA is frequently referred to as a “sequencing library”. Most of the next generation sequencing applications require the preparation of a sequencing library, recombinant DNA with specific adapters at 5′ and 3′ ends. For example, the Illumina sequencing workflow utilizes partially complementary adaptor oligonucleotides that are used for priming the PCR amplification and introducing the specific nucleotide sequences required for cluster generation by bridge PCR and facilitating the sequencing-by-synthesis reactions. This elaborate process includes physical, enzymatic and chemical manipulations and subsequent purifications of the sample DNA. For this purpose, sequencing library preparation protocol is labor intensive and the required amount of starting material is usually high. Time-consuming preparation protocol and requirement to start with micrograms of DNA reduce the throughput of genomic research projects and number of available samples. Furthermore, PCR-based library preparation involves clonal amplification reaction, which can introduce errors and skews the representation of the genomic elements.

### SUMMARY

Provided herein is a ligation-based method for preparing a template for sequencing, and a kit for performing the same. In certain embodiments, the method may comprise: a) digesting a sample comprising genomic DNA using a restriction enzyme to produce a digested sample; b) producing a circular nucleic acid comprising i. a splint oligonucleotide, ii. a vector oligonucleotide comprises a binding site for a first sequencing primer iii. a target genomic fragment, and iv. a duplex region in which the 5′ end of the vector oligonucleotide is ligatably adjacent to the 3′ end of the target genomic fragment, and the 3′ end of the vector is oligonucleotide is ligatably adjacent to the 5′ end of the target genomic fragment by: contacting, under hybridization conditions, the digested sample with: i. the vector oligonucleotide; and ii. the splint oligonucleotide, wherein the splint oligonucleotide comprises: a central region that hybridizes to the entirety of the vector oligonucleotide; a 5′ region that hybridizes to a first region in a target genomic fragment in the digested sample, and a 3′ region that hybridizes to a second region in the target genomic fragment; and, optionally enzymatic treatment remove any 5′ overhang from the target genomic fragment to make the 3′ end of the vector oligonucleotide ligatably adjacent to the 5′ end of the target genomic fragment; b) contacting the circular nucleic acid with a ligase, thereby ligating the 5′ end of the vector oligonucleotide to the 3′ end of the target genomic fragment and ligating the 3′ end of the vector oligonucleotide to the 5′ end of the target genomic fragment to produce a circular DNA molecule; c) separating the circular DNA molecule from the splint oligonucleotide; and d) sequencing the target genomic fragment of the circular DNA molecule using the first sequencing primer.

In certain embodiments, the method may comprise: a) contacting, under hybridization conditions, a target genomic fragment with: i. a vector oligonucleotide comprising binding sites for a sequencing primers and universal amplification sites; and ii. a splint oligonucleotide that hybridizes to the vector oligonucleotide and to the nucleotide sequences at the ends of the target genomic fragment, to produce a circular nucleic acid comprising a duplex region in which the 5′ end of the vector oligonucleotide is ligatably adjacent to the 3′ end of the target genomic fragment and the 3′ end of the vector oligonucleotide is ligatably adjacent to the 5′ end of the target genomic fragment; b) contacting the circular nucleic acid with a ligase, thereby ligating the 5′ end of the vector oligonucleotide to the 3′ end of the target genomic fragment and ligating the 3′ end of the vector oligonucleotide to the 5′ end of the target genomic fragment to produce a circular DNA molecule; and c) separating the circular DNA molecule from the splint oligonucleotide. The method may further include: d) sequencing the target genomic fragment of the circular DNA molecule using the end-specific sequencing primers.

The above-summarized method may be employed in a method of genome analysis that generally comprises: a) digesting a genome to produce a plurality of genomic fragments; b) contacting, under hybridization conditions, the plurality of genomic fragments with: i. a vector oligonucleotide comprising a binding site for a sequencing primer; and ii. a splint oligonucleotide that hybridizes to the vector oligonucleotide and to the nucleotide sequences at the ends of the a portion of the genomic fragments, to produce a plurality of circular nucleic acids comprising a duplex region in which the 5′ end of the vector oligonucleotide is ligatably adjacent to the 3′ end of a target genomic fragment and the 3′ end of the vector oligonucleotide is immediately adjacent to the 5′ end of the target genomic fragment; b) contacting the circular nucleic acid with a ligase, thereby ligating the 5′ end of the vector oligonucleotide to the 3′ end of the target genomic fragment and ligating the 3′ end of the vector oligonucleotide to the 5′ end of the target genomic fragment to produce a plurality of circular DNA molecules; c) separating the plurality of circular DNA molecule from the splint oligonucleotide. The method may further comprises: d) sequencing the target genomic fragments of the plurality of circular DNA molecules using the sequencing.

A kit is also provided. In certain embodiments, the kit comprises: i. a vector oligonucleotide comprising a first binding site for a sequencing primer and a second binding site for a second sequencing primer; and ii. a splint oligonucleotide that hybridizes to the vector oligonucleotide and to the nucleotide sequences at the ends of a plurality of restriction fragments in a mammalian genome or other organisms' genomes, wherein the vector and splint oligonucleotides are characterized in that, when hybridized with the restriction fragment, they produce a circular nucleic acid comprising a duplex region in at least the which the 5′ end of the vector oligonucleotide is ligatably adjacent to the 3′ end of the genomic fragment.

### BRIEF DESCRIPTION OF THE FIGURES

FIG. 1. Novel approaches for next-generation sequencing library preparation. A) Direct capture sequencing. B) Partitioned genome sequencing. C) Archived genome sequencing.

FIG. 2. Gel electrophoresis analyses of the direct capture sequencing library preparation steps. A) MseI digestion of NA18507 genomic DNA. B) Genomic circularization. C) Purification of the circles. D) PCR confirmation of the sequencing library. E) Sequencing is libraries prior to gel extraction. F) Sequencing libraries post gel extraction.

FIG. 3. End-sequencing targeted amplicons. A) Sequencing fold coverage of the APC gene exon 15 after 25 cycles of PCR. B) Sequencing fold coverage of the APC gene exon 15 by directly sequencing the captured circles. C) Sequencing fold coverage of individual captures.

FIG. 4. Gel electrophoresis analyses of the partitioned genome sequencing library preparation steps. A) Restriction enzyme digestion of lambda DNA. B) Titrating the template:adaptor ratio for ligation using MspI digested lambda DNA.

FIG. 5. Preparation of sequencing libraries using CRC cell line samples. MspI and HpaII restriction enzymes and 6:1 adaptor:DNA ratio were used in the ligation experiments. 300, 400 and 500 by fragments were size excised and 25 cycles of PCR was used to verify libraries.

FIG. 6. Single-strand template sequencing using degenerate oligonucleotide linker mediated adaptor ligation enforced PCR. A) Titration of template DNA and oligos. B) Library preparation using FFPE tissues. C) PCR amplified sequencing libraries. D) Gel purification of the sequencing libraries. E) Varying length degenerate regions of the linker oligonucleotides.

FIG. 7. Archived DNA sequencing. Genomic coverage of sequencing reads by DOLLM-PCR and conventional Illumina sample preparations. DNA copy number profile from a FFPE sample prepared using DOLLM-PCR.

FIG. 8. In-situ synthesis of oligonucleotides on microarray. A) Linear design. Sequence components for target DNA recognition, sequencing priming and library hybridization are synthesized in linear form and reagent amplification sites are incorporated in the synthesized oligos. B) Olignucleotide constructs for modular synthesis design. Three DNA components are synthesized. Highly complex set of oligonucleotides containing the target recognition sequences (labeled “Target circularization oligonucleotide”) can be synthesized on a microarray platform. “Adaptor circularization oligonucleotide” and “Adapter vector” can be synthesized in lower throughput system as the degree of complexity is equivalent to number of indexed/adapter functionalized reagent sets. C) Oligo circularization. Different indexing/adapter components are joined with the targeting oligonucleotides in a circularization reaction that makes possible of generating subset reagent sets that are indexed and complementary with various sequencing platforms. D) Amplification from circular template. E) Circularization of oligonucleotides.

FIG. 9. Purification of oligonucleotides after modular synthesis. Purification of the coding strand is done by using Uracil-incorporation during PCR amplification, nicking restriction enzyme digestion and denaturing PAGE purification.

FIGS. 10A-C. Targeted sequencing library preparation method. (a) Overview of the assay. (b) Specific preparation steps: (1) genomic DNA is digested using MseI restriction endonuclease. (2) Then, genomic DNA fragments are circularized using thermostable DNA ligase and Taq DNA polymerase for 5′ editing. Pool of oligonucleotides targeting 5′ and 3′ ends of the DNA fragments and vector oligonucleotide are used for targeted DNA capture. (3) After circularization, regular Illumina sequencing library can be prepared by PCR. (4) PCR amplified library fragments are similar to regular Illumina library constructs and anneal to immobilized primers on the flow cell. (5) Additionally, circular constructs can be directly sequenced as the adapted genomic DNA circles incorporate all DNA components required for library immobilization and sequencing. (c) Molecular structures of vector oligonucleotide and targeting oligonucleotides. SEQ ID NOS: 1 and 108.

FIGS. 11A-11D. Bioanalyzer analysis of the sequencing libraries. Targeted sequencing libraries were prepared by circularization in (a) 60 C, (b) 55 C, and (c) 50 C. (d) Electrogram.

FIGS. 12A-12B. Coverage of target region by end-sequencing genomic DNA. (a) 5′ ends of the targets are marked blue and 3′ ends of the targets are marked red. (b) 17 targeting is oligonucleotides (numbers 83-99) were designed to tile across exon 15 of the APC gene. Intermediate circularized genomic DNA is marked using black lines.

FIGS. 13A-13B. Uniformity of the coverage in (a) single-end sequencing libraries (experiments 2-5) and in (b) paired-end sequencing library (experiment 1) is presented. In the figures, median normalized sequencing fold-coverage (y-axis) is presented for each targeted position (y-axis). Targeted region in figure (a) was 4,410 bases and targeted region in figure (b) was 8,904 bases.

FIGS. 14C-14C. Relation between sequence read yield and (a) circle size, (b) high (G+C) contrent, and (c) low (G+C) content. Blue dots represent top performing oligos, red dots represent moderate performing oligonucleotides and green dots represent failed oligonucleotides.

FIG. 15. Schematic illustration of an exemplary embodiment of the method.

### DEFINITIONS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by is reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

The term “sample” as used herein relates to a material or mixture of materials, typically, although not necessarily, in liquid form, containing one or more analytes of interest.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “nucleic acid sample,” as used herein denotes a sample containing nucleic is acids.

The term “target polynucleotide,” as use herein, refers to a polynucleotide of interest under study. In certain embodiments, a target polynucleotide contains one or more sequences that are of interest and under study.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers. An oligonucleotide may be 10 to 20, 11 to 30,31 to 40,41 to 50, 51-60, 61 to 70, 71 to 80, 80 to 100, 100 to 150 or 150 to 200 nucleotides in length, for example.

The term “hybridization” refers to the process by which a strand of nucleic acid joins with a complementary strand through base pairing as known in the art. A nucleic acid is considered to be “Selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to generating one or more copies of a target nucleic acid, using the target nucleic acid as a template.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

As used herein, the term “Tₘ-matched” refers to a plurality of nucleic acid duplexes having Tₘs that are within a defined range.

The term “free in solution,” as used here, describes a molecule, such as a polynucleotide, that is not bound or tethered to another molecule.

The term “denaturing,” as used herein, refers to the separation of a nucleic acid duplex into two single strands.

The term “partitioning”, with respect to a genome, refers to the separation of one part of the genome from the remainder of the genome to produce a product that is isolated from the remainder of the genome. The term “partitioning” encompasses enriching.

The term “genomic region”, as used herein, refers to a region of a genome, e.g., an animal or plant genome such as the genome of a human, monkey, rat, fish or insect or plant. In certain cases, an oligonucleotide used in the method described herein may be designed using a reference genomic region, i.e., a genomic region of known nucleotide sequence, e.g., a chromosomal region whose sequence is deposited at NCBI's Genbank database or other database, for example. Such an oligonucleotide may be employed in an assay that uses a sample containing a test genome, where the test genome contains a binding site for the oligonucleotide.

The term “sequence-specific restriction endonuclease” or “restriction enzyme” refers to an enzyme that cleaves double-stranded DNA at a specific sequence to which the enzyme binds.

The term “affinity tag”, as used herein, refers to moiety that can be used to separate a molecule to which the affinity tag is attached from other molecules that do not contain the affinity tag. In certain cases, an “affinity tag” may bind to the “capture agent”, where the affinity tag specifically binds to the capture agent, thereby facilitating the separation of the molecule to which the affinity tag is attached from other molecules that do not contain the affinity tag.

With reference to two nucleic acid molecules or two nucleotides (i.e., a first oligonucleotide and a second oligonucleotide), the term “ligatably adjacent”, as used herein, refers to next to each other with no intervening nucleotides, such that the two nucleotides can be ligated to one another in the presence of a ligase. To be ligatable, one nucleotide will have a 3′ hydroxyl group and the other nucleotide will have a 5′ phosphate group.

The term “terminal nucleotide”, as used herein, refers to the nucleotide at either the 5′ or the 3′ end of a nucleic acid molecule. The nucleic acid molecule may be in double-stranded (i.e., duplexed) or in single-stranded form.

The term “ligating”, as used herein, refers to the enzymatically catalyzed joining of the terminal nucleotide at the 5′ end of a first DNA molecule to the terminal nucleotide at the 3′ end of a second DNA molecule.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

If two nucleic acids are “complementary”, each base of one of the nucleic acids base pairs with corresponding nucleotides in the other nucleic acid. The term “complementary” and “perfectly complementary” are used synonymously herein.

The term “digesting” is intended to indicate a process by which a nucleic acid is cleaved by a restriction enzyme. In order to digest a nucleic acid, a restriction enzyme and a nucleic acid containing a recognition site for the restriction enzyme are contacted under conditions suitable for the restriction enzyme to work. Conditions suitable for activity of commercially available restriction enzymes are known, and supplied with those enzymes upon purchase.

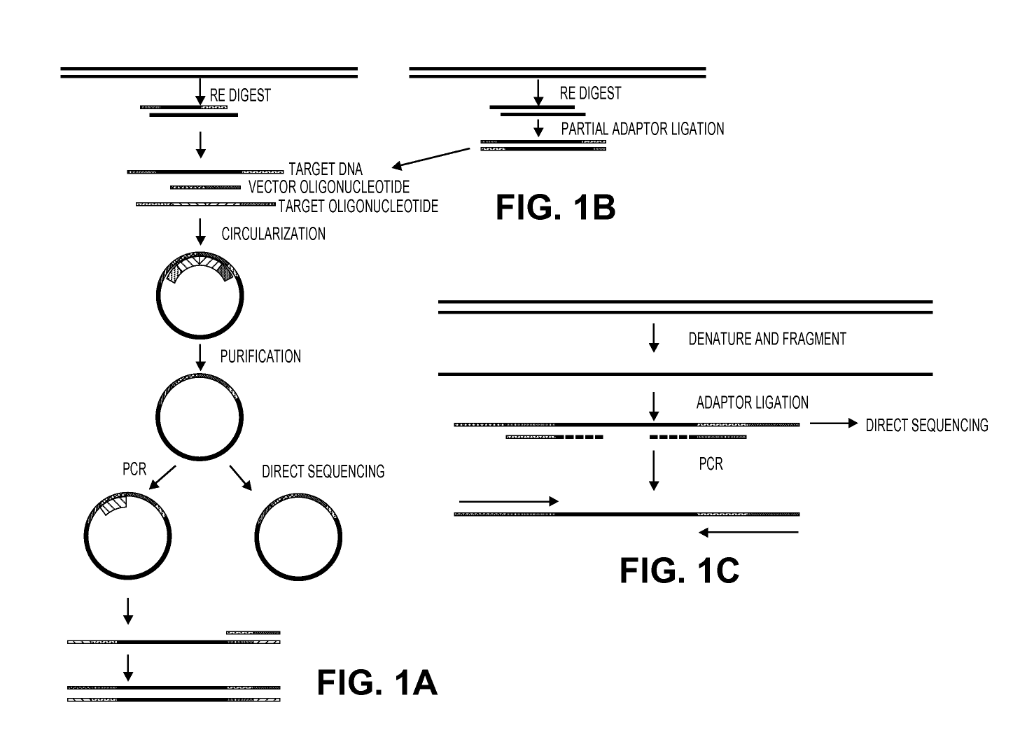

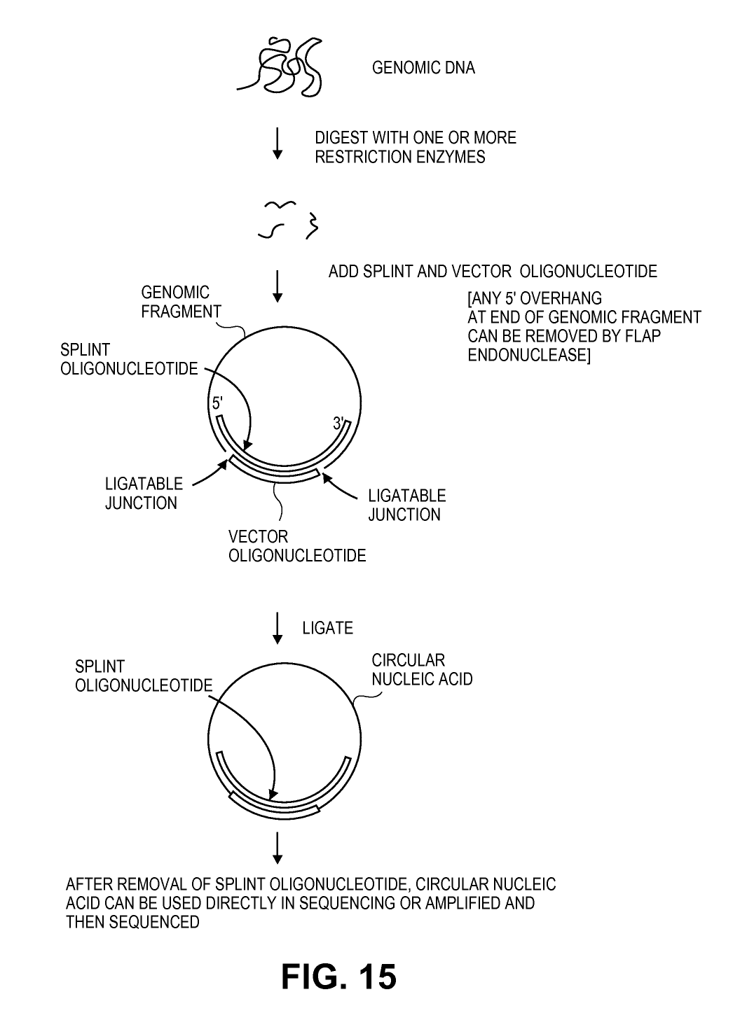

The term “vector oligonucleotide”, as used herein, refers to an oligonucleotide that is subsequently ligated to the target genomic fragment, as shown in FIGS. 1 and 15. The vector oligonucleotide contains binding sites for one or more sequencing primers and/or amplification primers, depending upon which specific method is employed. In certain cases, the vector oligonucleotide may contain sequences that are compatible with the sequences used in a next generation sequencing method such as that of Illumina, ABI, Roche, Pacific Biosciences, Ion Torrent and Helicos.

A “primer binding site” refers to a site to which a primer hybridizes in an oligonucleotide or a complementary strand thereof.

The term “splint oligonucleotide”, as used herein, refers to an oligonucleotide that, when hybridized to other polynucleotides, acts as a “splint” to position the polynucleotides next to one another so that they can be ligated together, as illustrated in FIG. 1. As illustrated in FIG. 1, a splint oligonucleotide may facilitate the production of a circular DNA molecule via two intramolecular ligations. Splint oligonucleotides may be referred to as “target oligonucleotides” in some parts of this disclosure.

The term “separating”, as used herein, refers to physical separation of two elements (e.g., by size or affinity, etc.) as well as degradation of one element, leaving the other intact.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, ABI, and Roche etc.

The term “linearizing” encompasses both enzymatic and chemical methods for breaking a strand of a circular DNA.

The term “circular nucleic acid” refers to covalently and non-covalently closed circles. A circular nucleic acid may be completely double stranded, completely single stranded or partially double stranded. A partially double stranded circular nucleic acid may contain one or more (e.g., 2, 3, 4, or more) single stranded regions separate the same number of double stranded regions.

The term “target genomic fragment” refers to both a nucleic acid fragment that is a direct product of fragmentation of a genome (i.e., without addition of adaptors to the ends of the fragment), and also to a nucleic acid fragment of a genome to which adaptors have been added. An oligonucleotide that hybridizes to a target genomic fragment to base-pair to the genome sequence or to the adaptors.

Other definitions of terms may appear throughout the specification.

### DETAILED DESCRIPTION OF EXEMPLARY EMBODIMENTS

As noted above, provided herein is a ligation-based method for preparing a template for sequencing, and a kit for performing the same. In certain embodiments, the method employs an oligonucleotide splint and vector to produce a circularized nucleic acid molecule containing binding sites for sequencing primers and clonal sequencing feature amplification and, in certain embodiments, binding sites for a pair of primers to that the template can be amplified by polymerase chain reaction. In an alternative embodiment and as will be described in greater detail below, a method is provided in which a splint oligonucleotide containing a region of degenerate nucleotide sequence is used to join a primer onto the ends of nucleic acid obtained from archived (e.g., formalin-fixed) material, e.g., a FFPE tissue biopsy. The methods and compositions described herein may be employed for re-sequencing applications, de novo sequencing applications and for sequencing of DNA fragments from archived material, for example.

Certain aspects of the method may be described with reference to FIG. 15. With is reference to FIG. 15, the first step of the method may comprise digesting a sample comprising genomic DNA using a restriction enzyme to produce a digested sample. Next, a circular nucleic acid is produced by contacting, under hybridization conditions, the digested sample with: i. a vector oligonucleotide; and ii. a splint oligonucleotide, wherein the splint oligonucleotide comprises: a central region that hybridizes to the entirety of the vector oligonucleotide; a 5′ region that hybridizes to a first region in a target genomic fragment in the digested sample, and a 3′ region that hybridizes to a second region in the target genomic fragment. This step may optionally comprises enzymatic treatment (e.g., with a flap endonuclease) to remove any 5′ overhang from the target genomic fragment to make the 3′ end of the vector oligonucleotide ligatably adjacent to the 5′ end of the target genomic fragment. As illustrated, the resultant circular nucleic acid comprising i. a splint oligonucleotide, ii. a vector oligonucleotide comprises a binding site for a first sequencing primer iii. a target genomic fragment, and iv. a duplex region in which the 5′ end of the vector oligonucleotide is ligatably adjacent to the 3′ end of the target genomic fragment, and the 3′ end of the vector oligonucleotide is ligatably adjacent to the 5′ end of the target genomic fragment. The circular nucleic acid is contacted with a ligase, thereby ligating the 5′ end of the vector oligonucleotide to the 3′ end of the target genomic fragment and ligating the 3′ end of the vector oligonucleotide to the 5′ end of the target genomic fragment to produce a circular DNA molecule. The method further comprises separating the circular DNA molecule from the splint oligonucleotide; and then sequencing the target genomic fragment of the circular DNA molecule using the first sequencing primer. The circular DNA molecule may be sequenced directly, or amplified prior to sequencing.

In particular embodiments, the vector oligonucleotide may further comprises a second binding site for a second sequencing primer and the sequencing step comprises sequencing the target genomic fragment of the circular DNA molecule using the first and second sequencing primers. The primer binding sites are generally compatible with the sequencing platform being used.

In some embodiments, prior to the sequencing step, the method may comprises amplifying the target genomic fragment of the circular DNA molecule by polymerase chain reaction (PCR) using a pair of primers that bind to primer sites that are also present in the vector oligonucleotide in addition to the sequencing primer site. The amplifying may be a bulk amplification in which the circular DNA molecules are amplified in a single reaction containing a plurality of the circular DNA molecules. In some cases the amplifying is clonal amplification in which the circular DNA molecules are amplified in separate reactions that are spatially distinct from one another, e.g., by bridge PCR or by emulsion PCR.

In some cases, the circular DNA molecule may be linearized prior to sequencing. The first steps of the method may be done in a single vessel without the addition of further reagents, and in certain cases the sequencing may be done in the absence of amplifying the circular DNA.

In some cases, the method may comprises enzymatic treatment to remove any 5′ overhang from the target genomic fragment to make the 3′ end of the vector oligonucleotide ligatably adjacent to the 5′ end of the target genomic fragment. In this step, a FLAP endonuclease, may be employed. The flap endonucleases may be of a eukaryotic, a prokaryotic, an archaea, or of a viral origin. In certain cases, FEN enzyme may be a Taq polymerase, flap endonuclease I, an N-terminal domain of DNA polymerase I or thermostable variants thereof.

In particular cases, steps c) and d) are done in a single vessel in which the genomic fragment, the vector oligonucleotide, the splint oligonucleotide and a thermostable ligase are thermally cycled through multiple rounds of a temperature suitable for denaturation and a temperature suitable for hybridization and ligation.

The method may be employed to isolate and provide the nucleotide sequence of a one or a plurality of known loci of a genome. The method may be employed to partition a genome.

As will be described in greater detail below, the sequencing may be done by any next generation sequencing method. Kits are also provided.

Certain aspects of the method are also described in FIG. 1. With reference to FIG. 1, certain embodiments of the method require, as noted above, contacting, under hybridization conditions, a target genomic fragment with a vector oligonucleotide and a splint oligonucleotide that hybridizes to the vector oligonucleotide and to the nucleotide sequences at the ends of the target genomic fragment. In this embodiment, the vector oligonucleotide contains at least one primer binding site for sequencing the target genomic fragment to which it ligates. In some embodiments and depending on the next generation sequencing platform for which the vector oligonucleotide is designed, the vector oligonucleotide may contain two primer binding sites (which prime in opposite directions) for sequencing from both ends of the genomic fragments to which the vector oligonucleotide is ligated. In addition, and depending on whether either a bulk or clonal amplification procedure is to be employed in the method, the vector oligonucleotide may further contain binding sites for a pair of PCR primers so that the genomic fragments to which the vector oligonucleotide is ligated can be amplified.

Since the vector oligonucleotide is to be ligated to a product of a restriction digestion or to adaptor ligated fragments, the vector oligonucleotide may have a 3′ hydroxyl group and a 5′ phosphate group, thereby allowing both ends of the vector oligonucleotide to be ligated to the genomic fragment (i.e., allowing the 5′ end of the genomic fragment, which may contain a 5′ phosphate, to be ligated to the 3′ of the vector oligonucleotide, which may contain a 3′ hydroxyl, and the 3′ of the genomic fragments, which may contain a 3′ hydroxyl, to be ligated to the 5′ end of the vector oligonucleotide, which may contain a 5′ phosphate). Depending on the sequencing platform to which the method is designed in conjunction with, the vector oligonucleotide may be at least 20 nt in length. In particular embodiments, the vector oligonucleotide is at least 50 nt in length (e.g., 50 nt to 150 nt in length), and the various primer binding sites in the vector oligonucleotide may be from 15 to 50 nt in length. Nucleotide sequences of exemplary vector oligonucleotides are set forth in the examples section of this disclosure.

The target oligonucleotide in the method, as illustrated in FIG. 1, is employed as a “splint” to facilitate the production of a circular nucleic acid comprising a duplex region in which the 5′ end of the vector oligonucleotide is ligatably adjacent to the 3′ end of the target genomic fragment and the 3′ end of the vector oligonucleotide is ligatably adjacent to the 5′ end of the target genomic fragment. As such and as illustrated in FIG. 1, the target oligonucleotide generally contains a central region (which is at least 15 nucleotides in from the ends of the oligonucleotide) that is complementary to the sequence of the vector oligonucleotide. As illustrated in FIG. 1, the regions flanking the central region of the target oligonucleotide are complementary to the ends of a target genomic fragment. The nucleotide sequence of the 5′ flanking region of a target oligonucleotide (which region may be of at least 15 nucleotides in length, e.g., 15 to 50 nucleotides) is complementary to the 3′ end of a target genomic fragment. Likewise, the nucleotide sequence of the 3′ flanking region of a target oligonucleotide (which region may be of at least 15 nucleotides in length, e.g., 15 to 50 nucleotides) is complementary to the 5′ end of a target genomic fragment. The vector oligonucleotide and target oligonucleotide are designed to produce a circular product when hybridized to a target genomic fragment, as shown in FIG. 1. Since the target oligonucleotide is not destined to be ligated to another nucleic acid, it may be designed so as to be unligatable. As such, in certain embodiments, the target oligonucleotide may have no 3′ hydroxyl and/or no 5′ phosphate groups, thereby preventing its ligation to other nucleic acids.

As noted above and as shown in FIG. 1 panel A, the target genomic fragment may be a restriction fragment of a genome that not adaptor ligated, in which case the flanking sequence of the target oligonucleotide may be designed to hybridize to specific restriction fragments of the genome. Depending on the desired complexity of the ligation, the method may be employed to capture one or more specific fragments from a genome, e.g., a single fragment or a plurality (at least 2, at least 5, at least 10, at least 20, at least 50, at least 100, at least 500, at least 1,000, at least 5,000, at least 10,000, at least 50,000 up to 100,000 or more) different fragments of a genome. In this embodiment, the method may employ a single vector oligonucleotide and multiple different target oligonucleotides that all contain a central region that hybridizes to the vector oligonucleotide and flanking sequences that hybridize to ends of genomic fragments, as desired. This embodiment is well suited for so-called “re-sequencing” applications in which the sequence of a reference genome is known and method is used to obtain the sequences for specific regions of a test genome, where the test genome is from the same species as the reference genome.

In other embodiments and as illustrated in FIG. 1 panel B, the target genomic fragment may be an adaptor-ligated restriction fragment of a genome, in which case the flanking sequence of the target oligonucleotide may be designed to hybridize to the adaptor sequences that have been ligated to the genomic fragment. In this embodiment, a single vector oligonucleotide and a single target oligonucleotide may be employed in the method to capture a desired population of genomic fragments. For example, the adaptor-ligated target genomic fragments may be size-selected prior to ligation. In other embodiments, the adaptor-ligated target genomic fragments are not size selected prior to ligation. This embodiment is well suited for so-called de novo applications in which the sequence of the target genome is not known and the method is used to obtain sequence information for the target genome.

After the oligonucleotides are annealed to one another, the resultant circular nucleic acid is contacted with a ligase, thereby ligating the 5′ end of the vector oligonucleotide to the 3′ end of the target genomic fragment and ligating the 3′ end of the vector oligonucleotide to the 5′ end of the target genomic fragment to produce a circular DNA molecule. The circular DNA molecule may be separated from the splint oligonucleotide after ligation, which may be done using, for example an exonuclease that would not degrade the circular DNA because it does not have a terminus. In a particular embodiment, the vector oligonucleotide may have an affinity tag that facilitates its purification from other material.

The resultant product, after its separation from the target oligonucleotide and optional cleavage to linearize the product (e.g., using a cleavable region in the vector oligonucleotide) may be directly employed in a sequence assay. In particular embodiments, product may be bulk amplified prior to sequencing using primers that bind to sites in the vector oligonucleotide.

In an alternative embodiment and as illustrated in FIG. 1C, an adaptor that is compatible with a next generation sequencing platform (i.e., an adaptor that contains binding sites for primers used in the platform) may be ligated to fragmented DNA, e.g., DNA obtained from an archived formalin fixed sample (e.g., an formalin fixed paraffin embedded FFPE sample) using a splint oligonucleotide that contains two regions: a first region, e.g., of 15 to 50 nucleotides, that is composed of a degenerate nucleotide sequence (i.e., where each nucleotide is N, where N is G, A, T or C) that base pairs with an end of the fragment, and a second region that is composed of a nucleotide sequence that base pairs with the adaptor. As illustrated in FIG. 1C, in this embodiment, a single splint oligonucleotide may be employed in conjunction with two vector oligonucleotides (one adapted to be ligated to only the 5′ end of the fragments, and the other adapted to be ligated to only the 3′ end of the fragments) to produce a double stranded product in which the fragment is ligatably adjacent to the vector oligonucleotides. As illustrated in FIG. 1C, after ligation, the linear product can be directly sequenced or amplified by PCR prior to sequencing.

The products described above may or may not be first amplified by PCR and then used as an input for a next generation sequence method. In certain cases and depending which platform is used, the products of the above may be applied to sequencing substrate, e.g., beads (454 or SOLID sequencing) or a flow cell (Illumina), and the products can be clonally amplification and sequenced.

The above described reagents, particularly the sequences of the vector oligonucleotides, are general compatible with one or more next-generation sequencing platforms. In certain embodiments, the products may be clonally amplified in vitro, e.g., using emulsion PCR or by bridge PCR, and then sequenced using, e.g., a reversible terminator method (Illumina and Helicos), by pyrosequencing (454) or by sequencing by ligation (SOLiD). Examples of such methods are described in the following references: Margulies et al (Genome sequencing in microfabricated high-density picolitre reactors”. Nature 2005 437: 376-80); Ronaghi et al (Real-time DNA sequencing using detection of pyrophosphate release Analytical Biochemistry 1996 242: 84-9); Shendure (Accurate Multiplex Polony Sequencing of an Evolved Bacterial Genome Science 2005 309: 1728); Imelfort et al (De novo sequencing of plant genomes using second-generation technologies Brief Bioinform. 2009 10:609-18); Fox et al (Applications of ultra-high-throughput sequencing. Methods Mol. Biol. 2009; 553:79-108); Appleby et al (New technologies for ultra-high throughput genotyping in plants. Methods Mol. Biol. 2009; 513:19-39) and Morozova (Applications of next-generation sequencing technologies in functional genomics. Genomics. 2008 92:255-64), which are incorporated by reference for the general descriptions of the methods and the particular steps of the methods, including all starting products, reagents, and final products for each of the steps.

The methods described above may be employed to investigate any genome, of known or unknown sequence, e.g., the genome of a plant (monocot or dicot), an animal such a vertebrate, e.g., a mammal (human, mouse, rat, etc), amphibian, reptile, fish, birds or invertebrate (such as an insect), or a microorganism such as a bacterium or yeast, etc.

Also provided by the present disclosure are kits for practicing the subject method as described above. The subject kit contains reagents for performing the method described above and in certain embodiments may contain i. a vector oligonucleotide comprising a first binding is site for a sequencing primer and a second binding site for a second sequencing primer; and ii. a splint oligonucleotide that hybridizes to the vector oligonucleotide and to the nucleotide sequences at the ends of a plurality of restriction fragments in a mammalian genome, wherein the vector and splint oligonucleotides are characterized in that, when hybridized with the restriction fragment, they produce a circular nucleic acid comprising a duplex region in which at lest the 5′ end of the vector oligonucleotide is ligatably adjacent to the 3′ end of the genomic fragment. In certain cases, the 3′ end of the vector oligonucleotide is also ligatably adjacent to the 5′ end of the genomic fragment. The kit may further include a ligase, adaptors, a restriction enzyme, flap endonuclease and/or other components described above.

In addition to above-mentioned components, the subject kit may further include instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In order to further illustrate the present invention, the following specific examples are given with the understanding that they are being offered to illustrate the present invention and should not be construed in any way as limiting its scope.

### EXAMPLES

### Materials and Methods I

Oligonucleotides. All oligonucleotides were synthesized at the Stanford Genome Technology Center (Stanford, Calif.). Direct capture sequencing oligonucleotides include 107 is target oligonucleotides (159-mers) that contain two hybridization regions (20 nt each) in the ends of the polymer and sequence components that correspond to forward (58 nt) and reverse (61 nt) Illumina paired-end adapters in the middle of the molecule (see Table 1 of 61/398,886). In addition, two 119 nt vector oligonucleotides were synthesized that are complementary to the middle portion of the targeting oligonucleotide and brings the ends of the targeted fragment in conjunction with DNA elements applied in the paired-end sequencing experiments. 5′ and 3′ ends of the targeting oliogonucleotides were blocked and did not contain phosphate or hydroxyl groups. In addition, targeting oligonucleotides contained 10 Uracils substitutions to facilitate fragmentation and purification of the oligo.

Genomic partitioning reagents included 13-16 nt long adaptor oligonucleotides, 119 nt long circularization oligonucleotide and 91 nt long vector oligonucleotides see (Table 2 of 61/398,886). One set of reagents was synthesized for MspI and HpaII assays and separate reagents were synthesized for CviQI and RsaI assays. 5′ end of the adaptor 1 oligonucleotides was blocked (no 5′ end PO₄ group) in order to inhibit adapter dimerization. Circularization oligonucleotides were blocked in 5′ and 3′ ends.

Single-strand DNA sequencing reagent set included: linker 1, linker 2, adapter 1 and adapter 2. 3′ end of the linker 1 contained 20 nt complementarity with the Illumina paired-end adaptor 1 and 5′ end had a 12 nt random degenerate sequence (see Table 3 of 61/398,886). Correspondingly, Linker 2 had degenerate sequence in the 3′ end and 20 nt region corresponding to adapter 2 sequence. Both linkers were blocked at 5′ and 3′ ends and 5′ end of the adapter 1 and 3′ end of the adapter 2 were blocked to inhibit any reactions between costruction oligos.

Samples. NA18507 and NA06695 samples were used in the approach validation experiments. A colon tissue sample was used in the single-strand sequencing experiment. Formalin-fixed paraffin-embedded sample (86-8047, NCCC) was used in the experiment.

Direct capture sequencing. 1.2 ug of genomic DNA from NA18507 (Coriell) was fragmented using MseI restriction enzyme (NEB) for 3 h in 37 C, followed by a heat inactivation of the enzyme for 20 min in 65 C. Target DNA was circularized in the presence of 107 oligonucletides targeting 10 cancer-related genes and vector oligonucleotide (Stanford Genome Technology Center, Stanford, Calif.). Circularization experiments were carried out using Ampligase thermostable ligase (Epicentre) and Taq (Invitrogen) for flap processing. After heat shock denaturing the sample in 95 C for 5 min, 15 circularization cycles (denature in 95 C for 2 min, hybridize in 60 C for 45 min and flap process for 15 minutes in 72 C) were performed. Circles were purified by degradation of the single-strand template and excess oligonucleotides using a mixture of Exonuclease I and III (NEB) and incubating the reaction in 37 C for 30 min, followed by heat inactivation of the enzymes (80 C, 20 min). Samples were further digested using Uracil-Excision enzyme (Epicentre). The circles were purified using Fermentas Gel Extraction and extracting 300-1200 bp fragments (direct sequencing) or PCR purification (amplification) and eluting in 30 ul. 10 ul of the purified circles were amplified using Phusion Hot Start DNA polymerase (Finnzymes, Finland) using Illumina paired-end library preparation primers and 25 PCR cycles (98 C, 10s; 65 C, 30s; 72 C, 15s) followed by extension step (72 C, 5 min). Amplified products (300 bp-1200 bp) were purified using Fermentas Gel Extraction kit. 10 pM of PCR amplified capture and 1.5 pM of direct capture were sequenced using Illumina Genome Analyzer II. Direct capture from 1 ug of starting material was introduced to the sequencing experiment. After sample dilution, 20% of the prepared sample (representing 200 ng of starting material) was hybridized in the flow cell. Paired-end sequencing of 36 bases was performed.

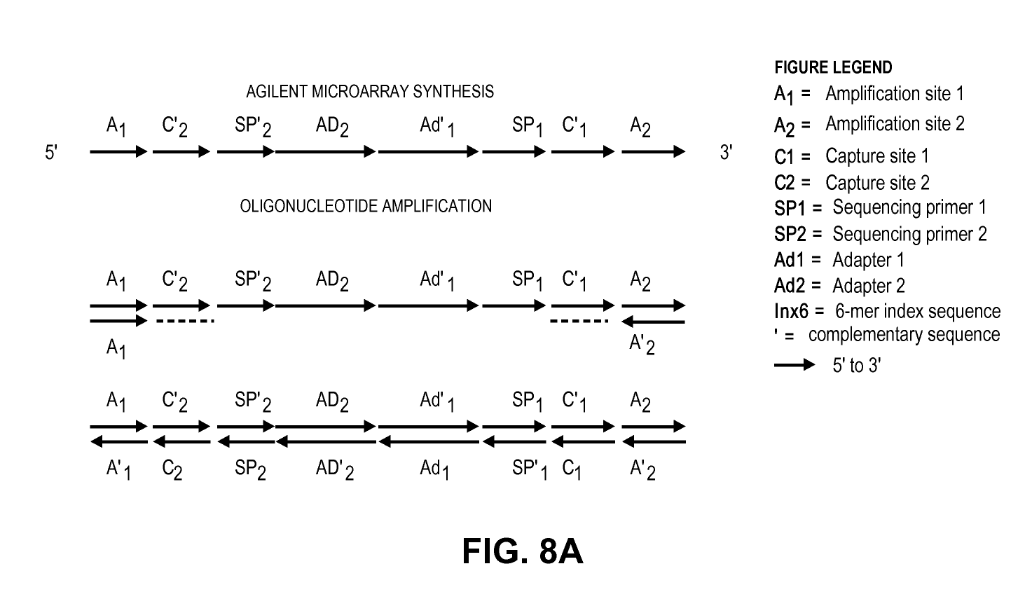

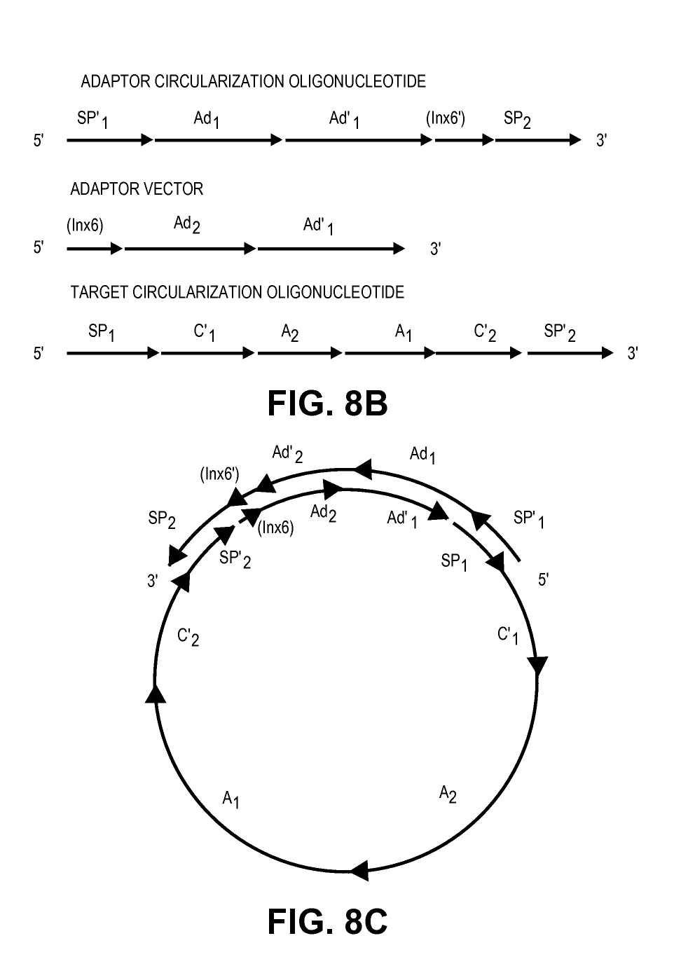

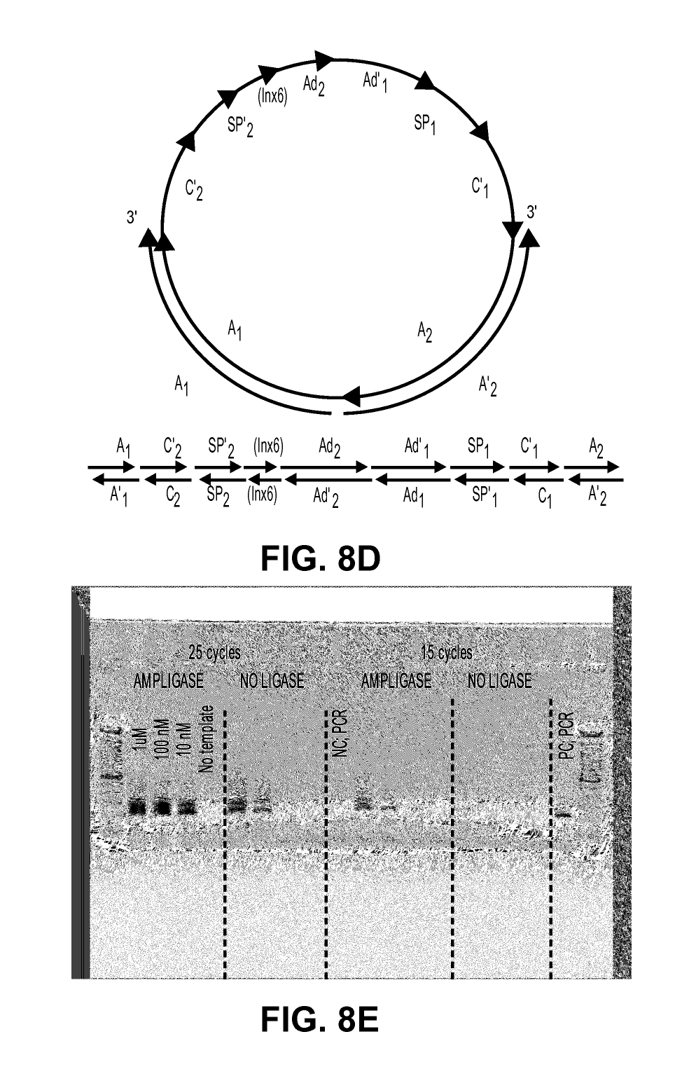

Modular oligonucleotide synthesis. Direct capture sequencing requires that capture oligonucleotides are synthesized in full and need to be readily functional in the assay as additional sequences can not be incorporated by PCR reaction. The aim of the protocol is to achieve highly multiplexed assays of tens of thousands of capture oligonucleotides. DNA microarray oligonucleotide production platforms, such as Agilent or NimleGen MAS, provide high-throughput oligonucleotide production capabilities. In-situ synthesis of oligonucleotides on a microarray surface can be used to achieve the highly complex oligonucleotide pools. However, the quantity of the oligonucleotides from the microarray synthesis is too low for direct use in the capture reactions. Therefore, amplification and purification schemes need to be incorporated in the microarray produce experiments (FIG. 8). In total, the synthetic oligonucleotides from the microarray need to be 199-mers. Furthermore, indexed reagents need to be synthesized on separate volumes and on multiple microarrays. In order to allow reagent indexing and synthesis of shorter oligonucleotides we have devised a modular method to generate oligonucleotides (FIG. 8).

All oligonucleotides were synthesized in the Stanford Genome Technology Center (see Table 4 of 61/398,886)). As a pilot experiment, 107 targeting oligonucleotides and oligos for 16-plex assay with 6-mer index sequences were generated. Modular design was applied to synthesize multiplexed reagents (FIG. 8). Three-component oligonucleotide system was circularized using 0.15 U of Ampligase (Epicentre) for 95 C, 5 min followed by 15 cycles of 95 C, 1 min; 60 C, 45 min; 72 C, 15 min. Splint oligo was fragmented using Uracil-DNA excision mix (37 C, 45 min; 95 C, 5 min) and samples were purified using CentriSpin CS-201 columns (Princeton Separations). Circularized template was used to amplify oligo contructs. Phusion Hot Start II DNA Polymerase, 0.5 uM primers and 800 nM dNTPs (200 nM each) were used in PCR (98 C, 30 s followed by 25 or 15 cycles of 98 C, 10 s; 50 C, 30 s; 72 C, 30 s.

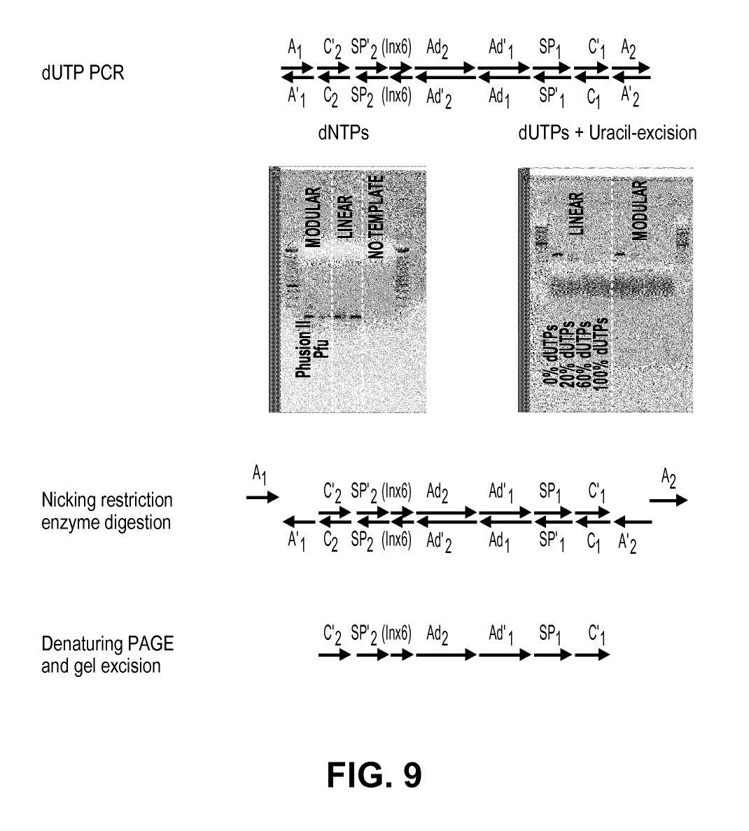

Purification scheme for the oligos (FIG. 9) includes PCR amplification using Cloned Pfu DNA polymerase (Invitrogen) in the presence of dUTPs. dUTPs are incorporated to the reagents as it is necessary in the purification of the oligos after genomic circularization. Amplification sites contain restriction enzyme cut sites for nicking endonucleases, Nb.BsrDI (New England BioLabs) and Nt.AlwI (New England BioLabs). After digestion, single-stand coding sequence of the capture oligo is purified using denaturing PAGE and gel excision.

Partitioned genome sequencing. Genomic DNA sample NA06995 was digested using MspI, HpaII, RsaI and CviQI restriction enzymes (NEB). 25 uM adapters were pre-annealed in 100 mM NaCl, 10 mM Tris-HCl pH 8 with overnight temperature ramp from 80 C to 4 C. Adapters were ligated to the ends of the restriction fragments using T4 DNA ligase (NEB). Adaptor:DNA ratio of 6:1 was used. 5′ ends of the adapters were phosphorylated using T4 polynucleotide kinase (NEB), 37 C for 30 min, followed by 65 C for 20 min. After adapter ligation, samples (300-450 bp fractions) were purified using Fermentas Gel Extraction kit. Adapted DNA fragments were circularized using targeting oligonucleotides and vector oligonucleotide. Ampligase (Epicentre) was used in the reaction and 15 ligation cycles (95 C, 2 min; 47 C, 45 min) were executed. After circularization, oligonucleotides were digested using Uracil-Excision (Epicentre) and purified using PCR purification kit (Qiagen). Illumina paired-end primers and Phusion Hot Start DNA polymerase were used to amplify and generate is sequencing library. Illumina paired-end sequencing was performed.

Archived genome sequencing. Genomic DNA was extracted from fresh frozen colon sample using DNeasy (Qiagen). DNA sample was fragmented using BioRuptor for 1 h and denatured by incubating in 95 C for 10 min. One 20 um sections of FFPE samples were lysed in 30 ul of WGA5 lysis buffer and heat shock (95 C, 10 min) was applied to resolve cross-linking. 100 ng of fragmented DNA and 5 or 2 ul of FFPE lysis were used as a template in the experiments. Linker oligonucleotides with 12 base degenerate regions and full Illumina adaptors were used in the ligation experiment. The ligation was performed using Ampligase thermostable ligase (Epicentre). After initial denature step (95 C, 5 min), 15 ligation cycles were run (95 C, 2 min; 72 C, 5 min; 65 C, 5 min; 60 C, 5 min; 55 C, 5 min; 50 C, 5 min; 45 C, 5 min; 40 C, 5 min; 35 C, 5 min; 30 C, 5 min). Fermentas Gel extraction (300-600 by fraction) was applied to purify the samples. After size fractionation Illumina paired-end primers and Phusion Hot Start DNA polymerase were used to generate sequencing libraries from the adaptor ligated material. Libraries were analyzed using Illumina paired-end sequencing.

### Results I

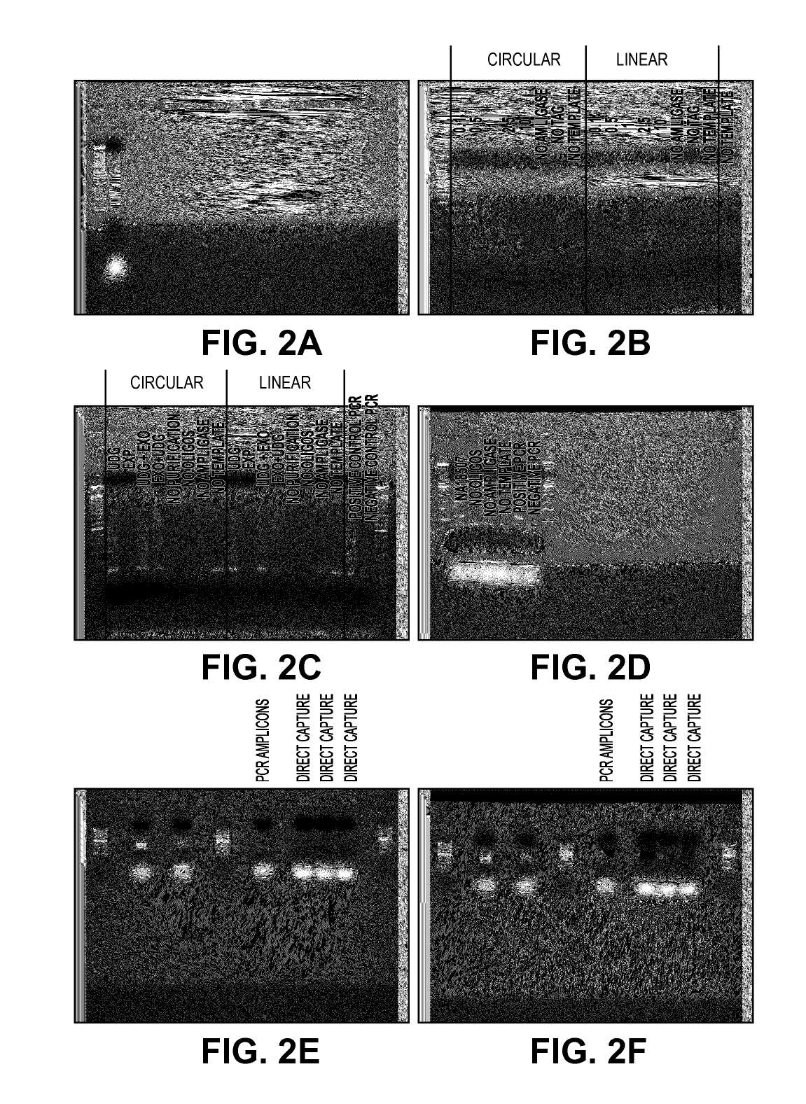

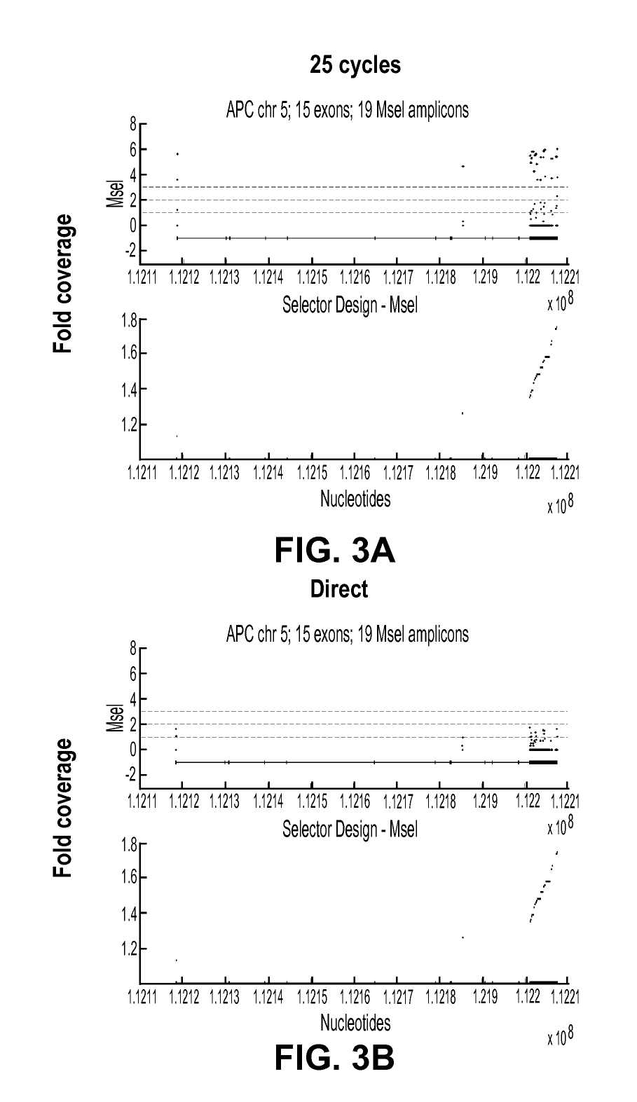

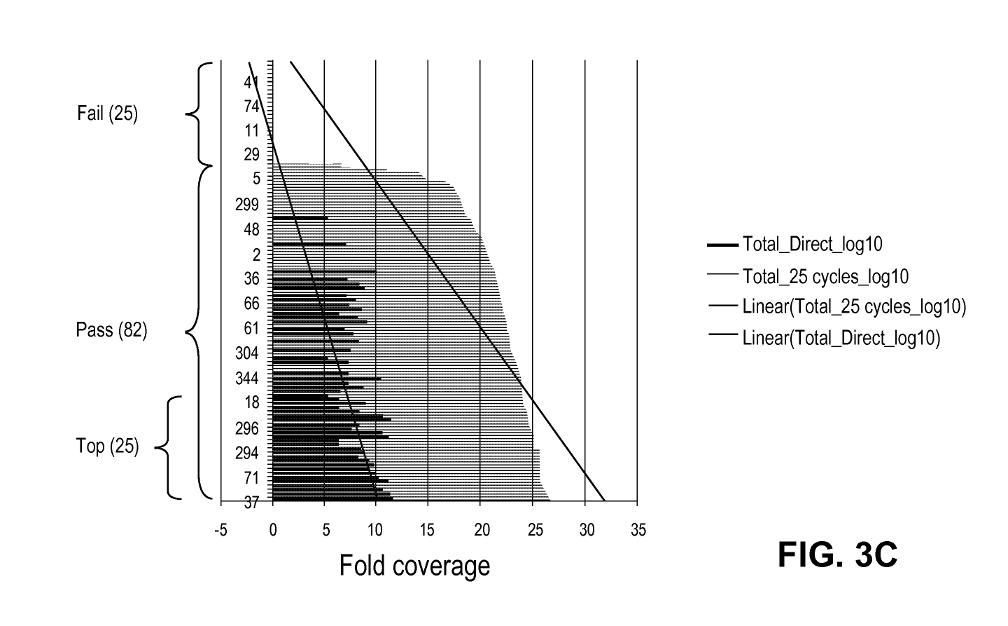

Direct capture sequencing. In this example, direct capture sequencing library preparation starts by MseI restriction enzyme digest. Gel electrophoresis analysis shows the fragmented DNA (FIG. 2A). After fragmentation circularization was carried out using different concentrations of the oligonucleotides (FIG. 2B). Increasing the oligo concentration results in deterioration of the signal and the optimal concentration of the oligos for initial optimization was 500 pM/oligo. No differences between circular and linear constructs were detected. Control samples (without oligos, ampligase, Taq or template DNA) yielded no amplicons. Different purification schemes were tested. Best purification was achieved using Exonuclease treatment followed by UDG excision (FIG. 2C). After circularization and purification, PCR confirmation was performed to verify proper library properties (FIG. 2D). Sequencing library preparation generated tractable pattern of different size amplicons without detectable background from the control samples (FIG. 2D). The sequencing library was prepared using 25 PCR cycles or directly extracting 300-1200 by circles from the gel (Figure 2E and F). Library concentrations were measured using SYBR Gold assay. PCR amplified library yielded 640 pM sample while direct capture sample was 30 pM.

Sequencing yielded 108 000 cluster/tile from the PCR amplicon end sequencing and direct capture sequencing yielded 2 500 clusters/tile. The sequences were shown to map to the ends of the amplicons. Same captured elements were shown to generate sequence data from the sample the was amplified 25 cycles and directly sequenced circles, indicating that direct capture sequencing is plausible (FIG. 2).

Modular oligonucleotide synthesis. Different concentrations of equimolar mixes of oligos were circularized and amplified. No ligase and no template samples were used as negative controls (FIG. 8E). 100 nM oligomix followed by 15 cycles of PCR was shown to generate specific 200 by band.

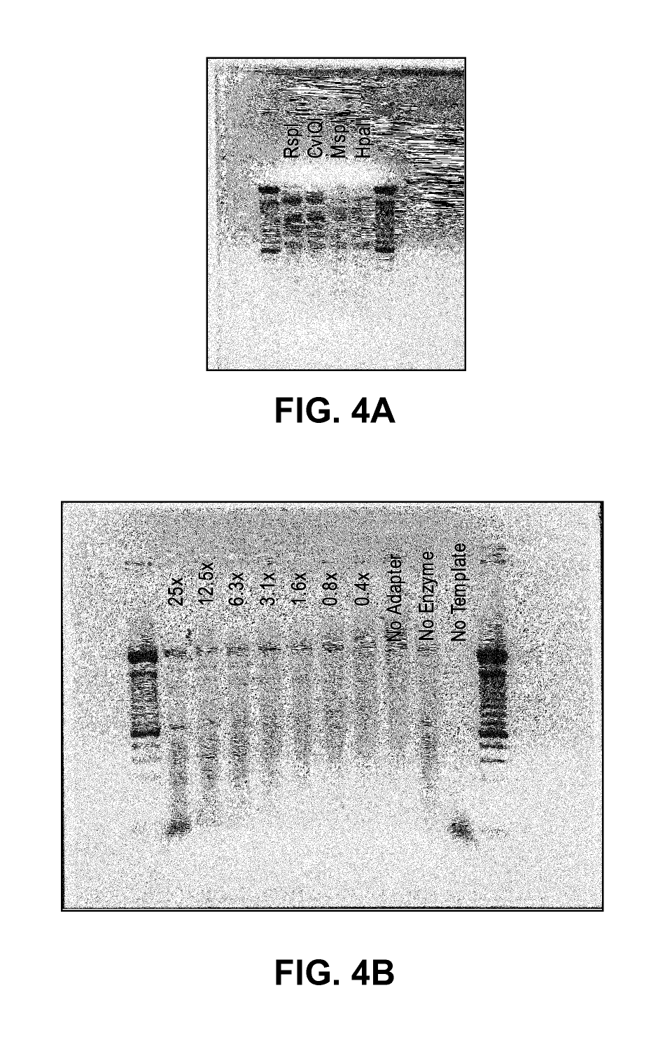

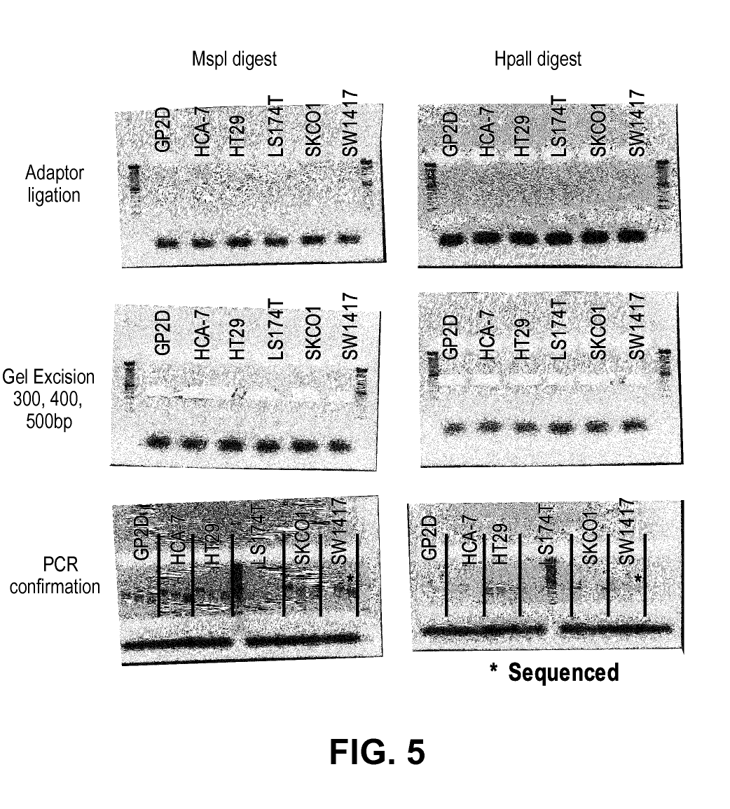

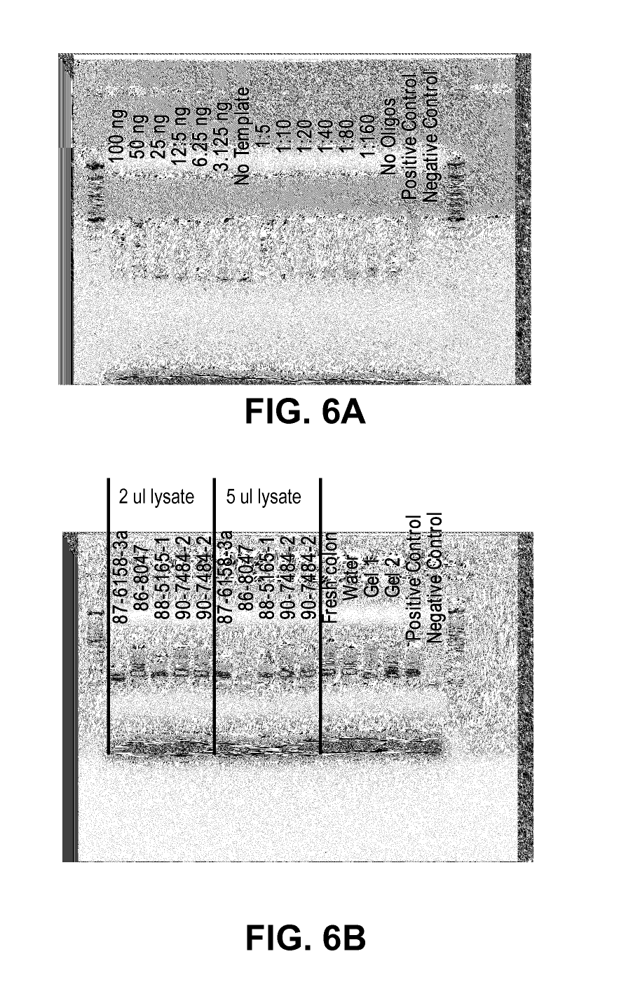

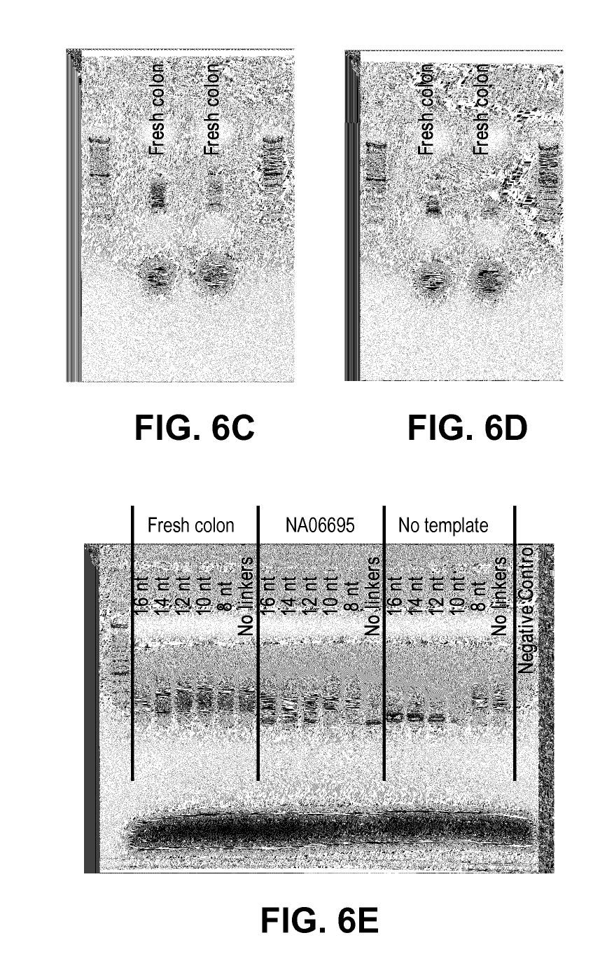

Partitioned genome sequencing. Lambda-phage DNA was used to set up the experiment conditions. Lambda genome DNA was digested using RsaI, HpaII, RspI and CviQI restriction enzymes and the amount of adaptor oligos in the ligation mix was titrated (FIG. 4). NA06695 (normal genomic DNA) and SW1417 (colorectal cancer cell line) and MspI and HpaII restriction digestions were used in the sequencing experiment (FIG. 5). Paired-end sequencing was performed using the libraries (FIG. 6).

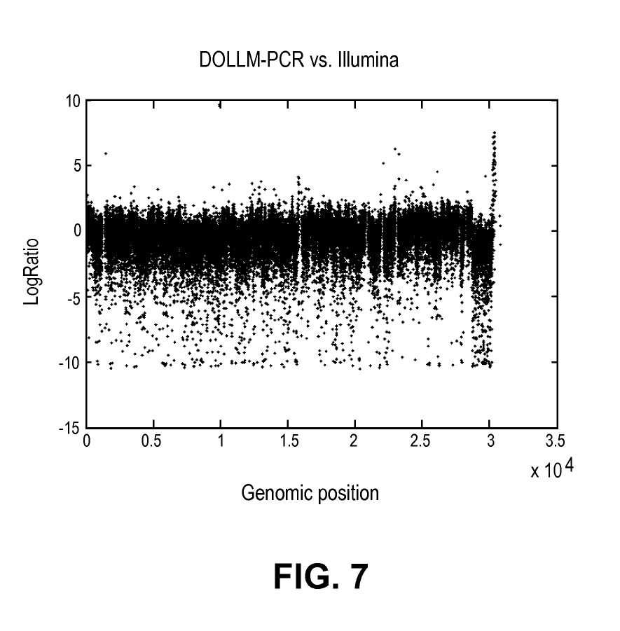

Archived genome sequencing. Sequencing library preparation specificity was tested by diluting the sample DNA and oligos. Library smear in the excised 400 bp region was visible using 6.25 ng of template DNA (FIG. 6A). 1:20 dilution was optimal when 50 ng of template DNA was prepared. FFPE tissues yielded libraries of varying quality (FIG. 6B). As a proof of concept, a fresh frozen CRC sample was fragmented, heat shock denatured and 100 ng of genomic was prepared for sequencing. 25 PCR cycles were ran using 10 ul of the adapted DNA (⅓ of the library) (FIG. 6C), 300-450 bp fraction was excised from the gel (FIG. 6D) and purified, yielding 30 ul of 5.0 pM sequencing library. Different lengths of the degenerate region (8-16 nt) were tested. 10 or 12 nucleotide random sequence provided best yields (FIG. 6E). Paired-end sequencing of 12 pM from the fresh DNA sample yielded 34.6 million paired reads and FFPE sample generated 30 million paired reads. On average 50% of all reads could be aligned to the human genome. When the distribution of sequence reads from the fresh DNA sample was compared to same sample prepared using conventional Illumina protocol, we observed that the genomic coverage of the reads was generally equal but some chromosomal regions were under represented (FIG. 7). In addition, unbalanced representation of sex chromosomes due to the male vs. female comparison was observed.

The assays described above can be used to prepare sequencing libraries of targeted, partitioned and archived genomic DNA content. The adapted DNA molecules are directional, in correct orientation and sequencable using standard Illumina sequencing reagents, and can be readily adapted for use in other next generation sequencing methods. The proposed methods enable preparation of next-generation sequencing libraries substantially faster from nanogram amounts and without PCR amplification. Our results demonstrate the proof-of-concept of the approaches and general applicability in deep resequencing of targeted DNA, partitioned genomes and formalin-fixed paraffin-embedded samples.

### Materials and Methods II

Oligonucleotides. Exons of 10 cancer-related genes were selected for targeting. Capture oligonucleotides include 107 target oligonucleotides (159-mers; see below)) that contain two hybridization regions (20 nt each) in the ends of the oligonucleotide and sequence components that correspond to forward (58 nt) and reverse (61 nt) Illumina paired-end adapters. At least one of the targeting arms is coincides with the last 20b of an MseI restriction fragment. When only one of the targeting arms is adjacent to a restriction site, the other end of the captured DNA strand forms a 5′P extension which is degraded during the circularization reaction by the 5′-exonuclease activity of Taq Polymerase (Lyamychev et al. 1993, v260, p778), thereby allowing Ampligase to form a single stranded circle. Targeting arms were positioned in SNP-free regions as defined by a lack of overlap with dbSNP129. In addition, 119 nt vector oligonucleotide was synthesized (see below). Vector oligonucleotide is complementary to the targeting oligonucleotides. 5′ and 3′ ends of the targeting oliogonucleotides were blocked and did not contain phosphate or hydroxyl groups. In addition, targeting oligonucleotides contained 10 Uracils substitutions to facilitate fragmentation and purification of the oligo. All oligonucleotides were synthesized at the Stanford Genome Technology Center (Stanford, Calif.).

Targeted genomic circularization. Genomic DNA obtained from NA18507 (Coriell Institute) was used for demonstration of targeted circularization based sequencing library preparation. 1 μg of genomic DNA from NA18507 (Coriell) was fragmented using MseI restriction endonuclease (NEB) for 3 hours in 37° C., followed by a heat inactivation of the enzyme for 20 min in 65° C. MseI digested genomic DNA was circularized in the presence of pool of 107 genomic circularization oligonucleotides (50 pM/oligo) and vector oligonucleotide (10 nM). Circularization experiments were carried out using Ampligase thermostable ligase (Epicentre) and Taq DNA polymerase (Invitrogen) was used for 5′ flap processing. After heat shock denaturation of the sample in 95° C. for 5 min, 15 circularization cycles (denature in 95° C. for 2 min, hybridize in 60° C. for 45 min and flap processing in 72° C. for 15 minutes) were performed.

Purification of captured genomic circles. Circles were purified by degradation of the single-strand template and excess linear oligonucleotides using a mixture of Exonuclease I and III exonuclease enzymes (NEB) and incubating the reaction in 37° C. for 30 min, followed by heat inactivation of the enzymes (80° C., 20 min). Samples were further digested using Uracil-Excision enzyme (Epicentre) to fragment the targeting oligonucleotides. Size fractions corresponding to 300-1200 bases were extracted from circularized DNA preparations using Gel Extraction purification (Epicentre). Purified circles were eluted to 30 μl.

Preparation of the amplification libraries. 10 μl of the purified circles were amplified using Phusion Hot Start DNA polymerase (Finnzymes, Finland) and general Illumina paired-end library preparation primers. 25 PCR cycles (98 C, 10s; 65 C, 30s; 72 C, 15s) followed by an extension step (72 C, 5 min) were run. Amplified products (300 bp-1200 bp) were purified using Fermentas Gel Extraction kit.

Sequencing. 10 pM of PCR amplified library and 1.5 pM of circularized DNA were sequenced using Illumina Genome Analyzer II. Circular library obtained from 1 μg of starting material was introduced to the sequencing experiment. After sample dilution using hybridization buffer, 20% of the prepared sample (representing 200 ng of starting material) was hybridized in the flow cell. Paired-end sequencing of 42 bases was performed using Illumina Genome Analyzer IIx.

Data analysis. Sequence reads were aligned to the human genome version hg17 using the ELAND software. We used a sub-reference of 102,488 bases, which encompassed the genomic DNA regions of the circularized targets. After alignment, depth matrices were constructed, where each row represented a single position in the sub-reference. We defined the target region by location of the target specific sites and delineating the 42 base regions (length of the sequencing reads) that corresponded to end-sequenced portions of the captured fragments. In paired-end experiment the target region contained both ends of the circularized fragments, while single-read sequencing targeted only 3′ ends of the circularized fragments. To assess the specificity of the capture we compared the numbers of sequence reads mapping within and outside the target region. To illustrate the uniformity of the assay, we counted the reads that aligned perfectly with the specific capture sequences. Read counts were then sorted and normalized using the median sequence yield value from each experiment. To evaluate the properties of the targeting oligonucleotides the genomic distance between the target specific sites measured the circle size. In addition, guanine and cytosine proportion within the target sites were determined. A single targeting oligonucleotide contained two target specific sites and each site was analyzed separately. To analyze the annealing properties during circularization-hybridization reaction, we classified target specific sites within a single targeting oligonucleotide as high or low (G+C). We then plotted circle sizes and (G+C) proportions with the sequence yields for each oligonucleotide. Finally, we performed genotyping by majority voting.

### Results II

Method for Targeted Sequencing Library Preparation by Genomic Circularization

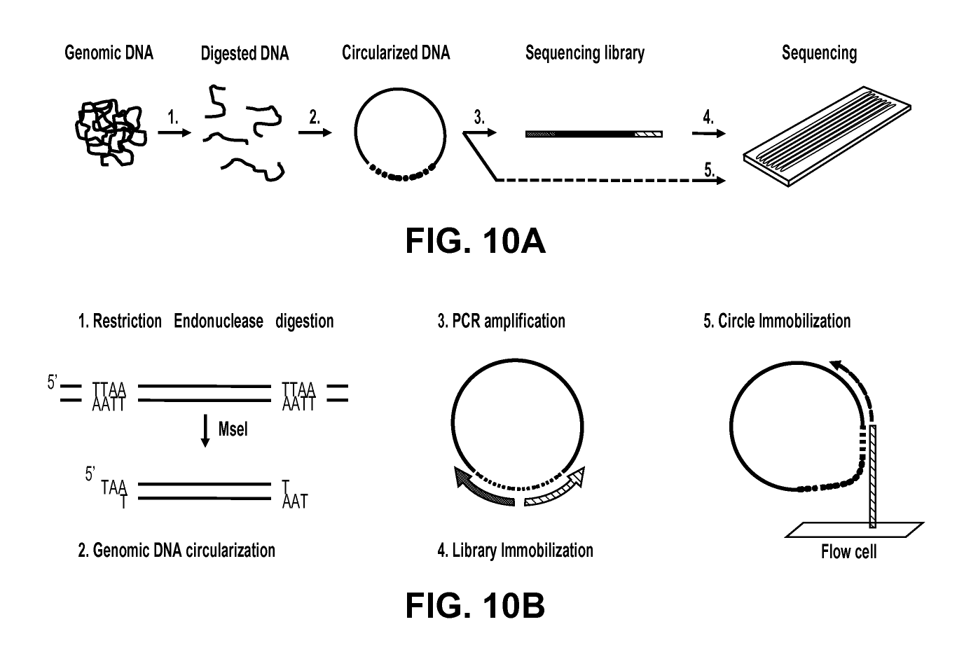

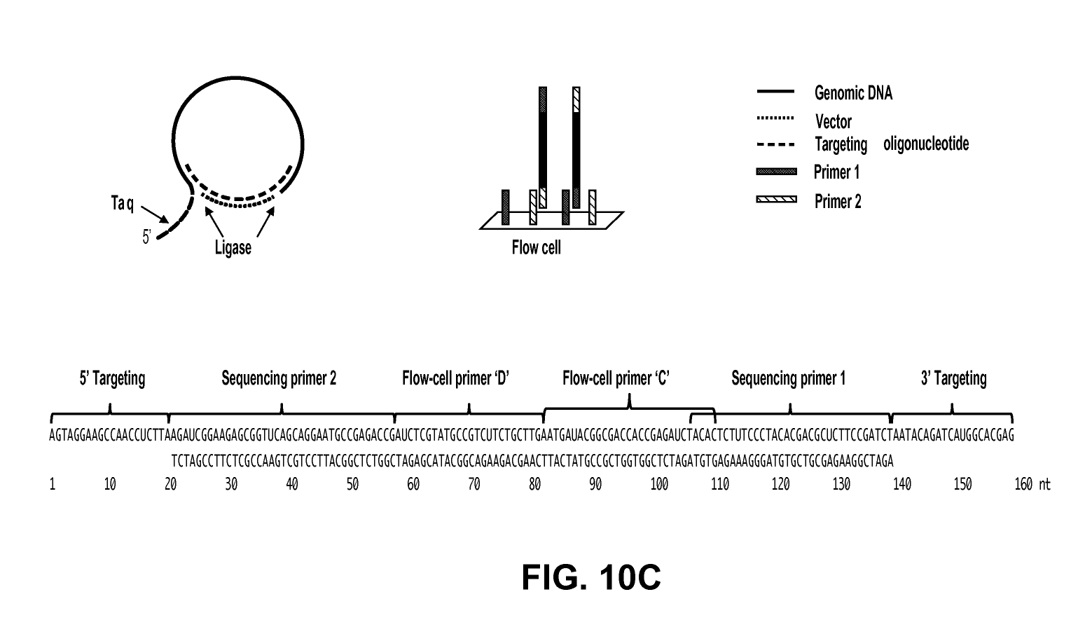

The method provides an approach for preparing next generation sequencing (NGS) libraries of targeted DNA content (FIG. 10a). First, we digested genomic DNA using MseI restriction endonuclease (FIG. 10b). Then, we used a pool of targeting oligonucleotides as splints and circularized the genomic DNA fragments by double-ended ligation to a common vector oligonucleotide. We carried out 15 circularization cycles using a thermostable ligase. While 3′ end of the targeted genomic DNA fragment has to align perfectly with the targeting and vector oligonucleotides, 5′ end of the fragment may contain an overhang. We used Taq DNA polymerase to process the 5′ overhang during the circularization reaction. In our assay, genomic DNA sites next to the 3′ end and next to or in proximity of the 5′ end of the circularized fragments are targeted. The common vector incorporates sites for primers that are required for sequencing (FIG. 10c). After purification, circles can be amplified using general IIlumina library preparation primers or directly sequenced using the IIlumina Genome Analyzer IIx.

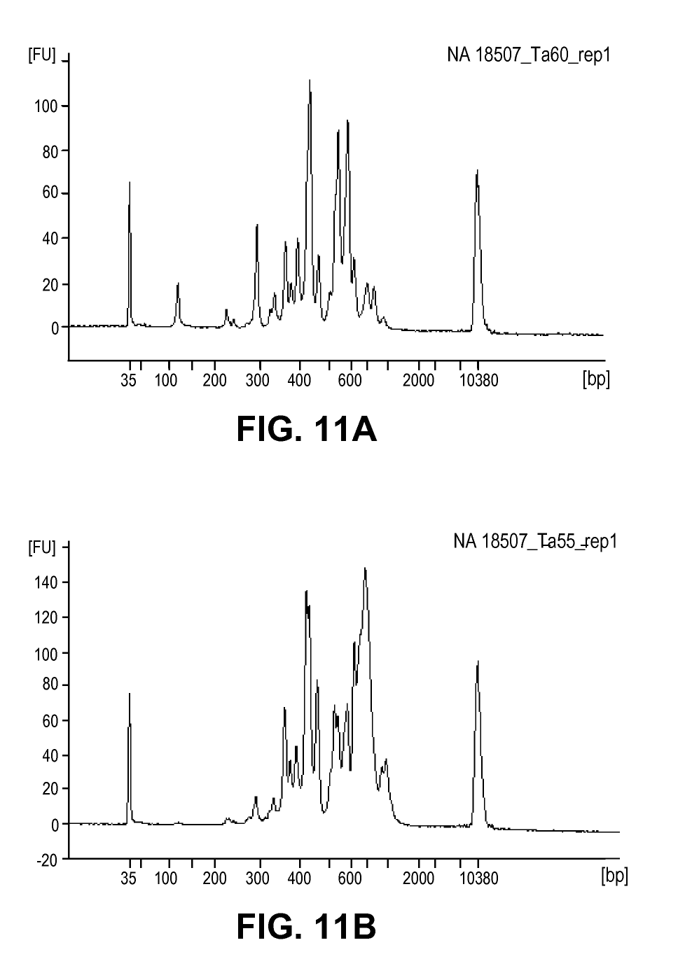

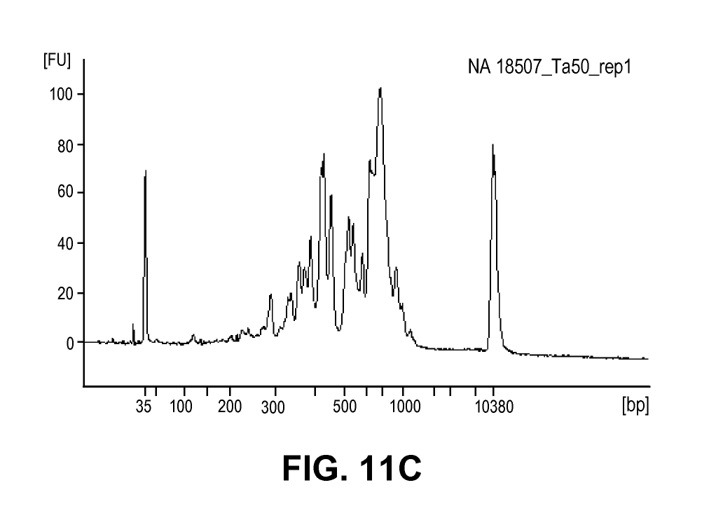

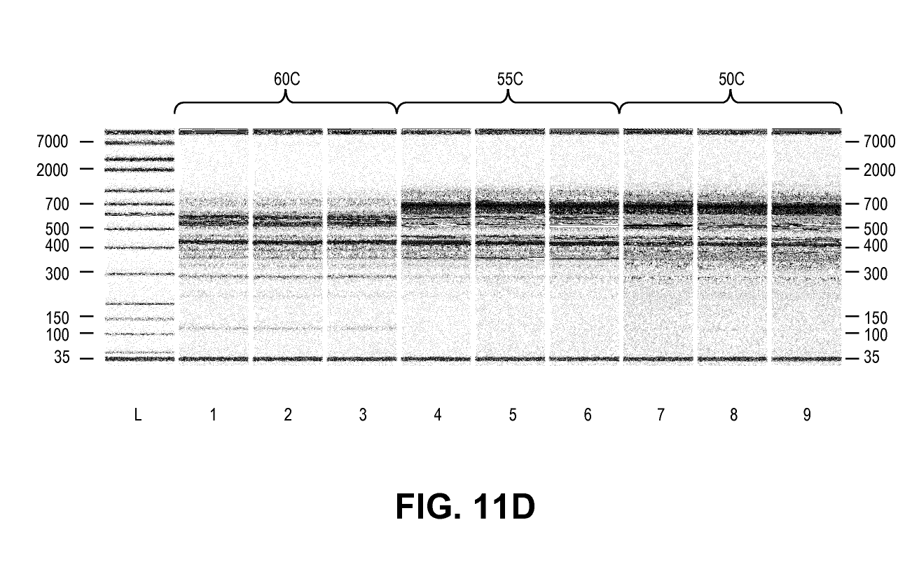

As a proof of concept, 107 oligonucleotides were designed to capture exonic regions of 10 cancer-related genes. The sequences of the oligonucleotides are provided in the sequence listing. Details of where the oligonucleotides bind are shown in Table 2. Targeted sequencing libraries were prepared from human genomic DNA (NA18507). For demonstration of differences between capture condition we prepared targeted sequencing libraries by hybridizing targeting oligonucleotides in 60, 55 and 50° C. during circularization reactions. Analysis of the libraries revealed that different hybridization conditions during circularization affect the fragment size pattern of the captured circles (FIG. 11). Five independent targeted libraries (experiments 1-5) were sequenced using the IIlumina system (Table 1). Each experiment was sequenced on a single IIlumina GAIIx lane. Sequence quality from PCR amplified libraries was high, as up to 93% of reads mapped to human genome. Single molecule experiment yielded less mappable sequence data due to small number of molecular targets in the human genomic DNA sample. However, our data demonstrates that it is possible to directly sequence circularized DNA without PCR amplification.

TABLE 1
Sequencing results.

| Experiment | 1 | 2 | 3 | 4 | 5 |
|---|---|---|---|---|---|
| Hybridization temperature (° C.) | 60 | 60 | 55 | 50 | 55 |
| Number of PCR cycles | 25 | 25 | 25 | 25 | Direct |
| Sequencing read length | 42 by 42 | 42 | 42 | 42 | 42 |
| Total reads | 34,081,017 | 12,542,683 | 15,605,713 | 12,435,664 | 1,232,093 |
| Mapped reads ^{a} | 31,655,174 | 8,576,700 | 13,415,111 | 7,381,662 | 11,726 |
| Captured on-target reads used | 31,324,396 | 7,560,090 | 11,105,527 | 6,330,012 | 8,488 |
| for genotyping ^{b, c} | | | | | |
| Captured off-target reads | 330,778 | 1,016,610 | 2,309,584 | 1,051,650 | 3,238 |
| On-target region (bases) ^{c} | 8,904 | 4,410 | 4,410 | 4,410 | 4,410 |
| Captured on-target region (bases) ^{c, d} | 6,670 | 3,145 | 3,340 | 3,044 | 2,809 |
| Captured on-target region used | 6,502 | 2,932 | 3,128 | 2,961 | 2,160 |
| for genotyping (bases) ^{b, c} | | | | | |
| Average sequence fold-coverage | 149,164 | 72,001 | 105,767 | 60,286 | 81 |
| on on-target region | | | | | |
| Non-reference positions on | 14 | 5 | 15 | 25 | 0 |
| on-target region ^{b, c, e} | | | | | |
| Concordance rate | 99.8% | 99.9% | 99.7% | 99.4% | 100.0% |

^{a} ELAND alignment using sub-reference (102,488 bases).
^{b} Sequencing fold-coverage >30.
^{c} Compilation of 42-base end-sequences from circularized targets.
^{d} Sequencing fold-coverage >1.
^{e} Sequence fold-coverage matrix and majority voting scheme.

Seamless integration of sequencing library preparation and target enrichment has many advantages. By streamlining the targeted resequencing process, the preparation time can be reduced to one day. In addition, fewer enzymatic reactions and purification steps suggest that significantly smaller samples and less starting material can be used for the analysis. Another major advantage is that amplification of the library is not necessary since the circular intermediate already incorporates all DNA components required for sequencing. Obviating the use of amplification omitted synthesis artifacts associated with the use of DNA polymerases.

### Assessment of the Capture Coverage

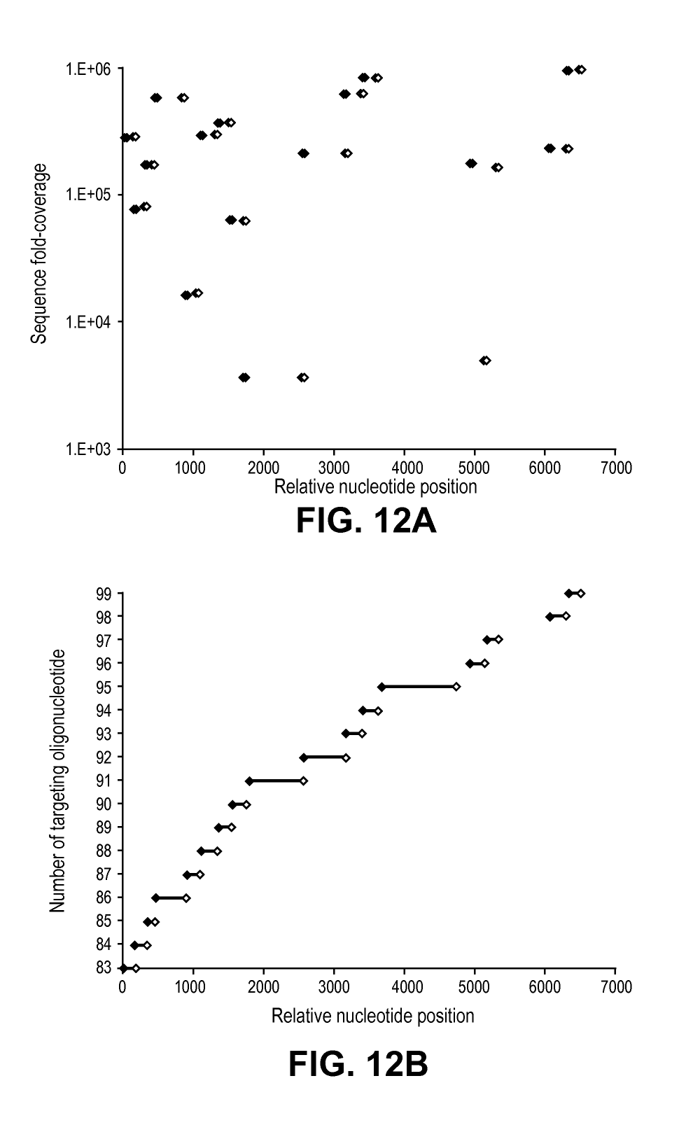

As an example of typical coverage profile, we present sequencing data from exon 15 of the APC gene (FIG. 12a). By design, our assay mediates end-sequencing of the targeted fragments and FIG. 12 shows how captured sequences map to the ends of the circularized amplicons. To illustrate the sequencing coverage we tiled genomic circularization probes across 6,523 by region in APC (FIG. 12b). These targeted sites were sequenced at high is fold-coverage compared to adjacent regions. Average sequencing fold-coverage for targeted regions were in the range of tens of thousands for the PCR amplified libraries. Average sequencing fold-coverage for directly sequenced circles was over 80.

To evaluate the specificity of targeting, the numbers of sequences derived within and outside of the targeted regions were compared. For paired-end sequencing, our target region encompassed 8,904 bases, defined by the read length (42 bases) and the end-sequenced portion of the circularized targets (Table 1). With paired-end sequencing of PCR amplified library (experiment 1), high on-target specificity was observed, as only 1% of the mapped reads were outside of the targeted regions. With single-end reads (see experiments 2-5), the target region was approximately half, 4,410 bases, because only 3′ ends of the captured circles were sequenced. Single read PCR amplified experiments (2-4) showed slightly higher off-target rate than paired-end sequencing. Direct sequencing of the circularized DNA without PCR amplification yielded the most off-target sequences (28). The obtained sequences were highly specific because sequencing adapter ligation is an integral part of the targeted capture process and dual-end hybridization is required for successful circle formation.

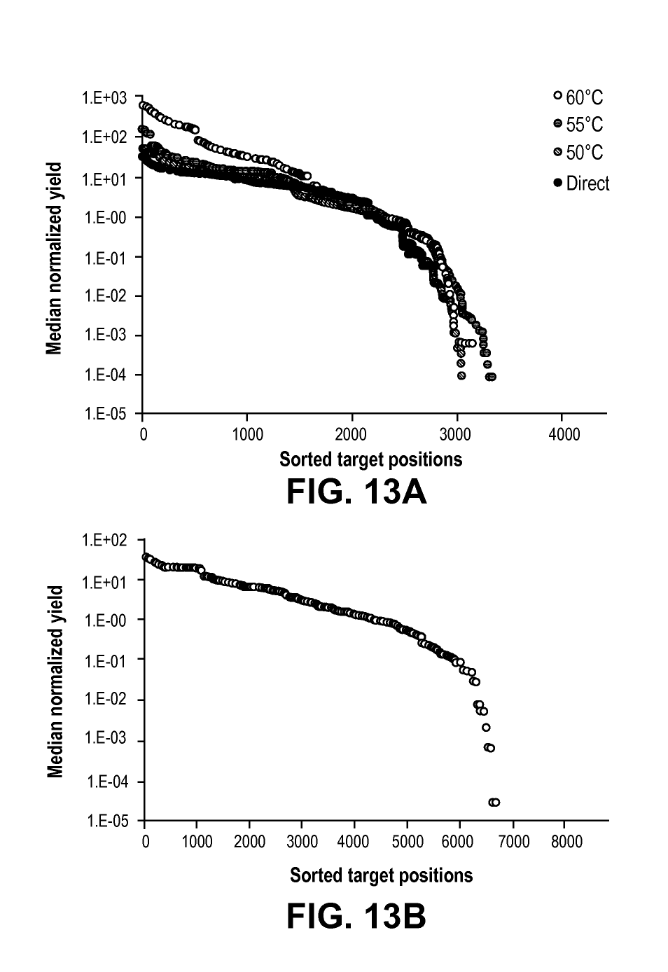

The regional coverage of the targets was analyzed. It was determined that 75% of the target region was captured at least once and 73% of the targeted bases were captured with fold-coverage above 30 by paired-end sequencing of the PCR amplified library (Table 1). Similarly, 64% or 49% of the target region was covered at least once or over 30-fold, respectively, when amplification-free circular library (experiment 5) was sequenced. The difference in coverage between amplicon and single molecule sequencing reflects the overall lower sequencing depth of direct circular library. In addition, we showed that hybridization in 55° C. resulted in higher coverage (76%) compared to target coverage by circularization in 60° C. or 50° C. (71% and 69%, respectively). The intent of this study was to explore the molecular properties of the assay. Therefore, we did not optimize any parameters that might affect capture efficiency, such as hybridization conditions or circle size, suggesting that observed holes in the target coverage reflect these conscious shortcomings of the oligonucleotide design. To assess the uniformity of the capture, oligonucleotides were sorted based on the capture yields. The yield distributions are presented in FIG. 13. We compared hybridization temperatures of 50, 55 and 60° C. in order to identify optimal circularization conditions for our complex targeting oligonucleotide pool. Our data shows that lower hybridization temperature during circularization results in more even coverage between different targeting oligonucleotides (FIG. 13a). Interestingly, the most even coverage was observed in directly sequenced sample, suggesting that PCR amplification is responsible for at least part of the differnces in capture efficiency. The uniformity of the coverage from paired-end data (experiment 1) was also assessed by binning the mated sequencing reads for each capture oligonucleotide (FIG. 13b). These data suggest that optimal circularization conditions and ability to perform single molecule capture improve the uniformity of the targeting assay. Our initial proof-of-concept demonstration encompassed at least 109 genomic target regions. However, there are numerous opportunities for increasing the throughput of the assay. For example, the complexity of the assay and the size of the target region can be increased by using multiple restriction endonucleases in the genomic fragmentation and by adding more targeting oligonucleotides. Especially in the amplification-free sequencing approach, higher complexity of the targeting oligonucleotide library is required for efficient use of sequencing capacity.

### Evaluation of Properties of the Targeting Oligonucleotides that Affect Sequence Capture Yield

Holes in the coverage and skewness of the capture uniformity are directly associated with the inefficiencies of the specific targeting oligonucleotides. Two possible failure modes were identified: target circularization fails due to unfavorable properties of the targeting sites and size of the captured template is unsuitable for sequencing. Optimizing the molecular properties of the targeting oligonucleotides may improve the assay. Since the first 20 bases of the sequencing reads are complementary to the target specific sites, individual targeting oligonucleotide species can be directly linked with sequencing data. With paired-end analysis the confidence of linking sequencing data to specific oligonucleotides increases substantially because of the dual-end specificity required for targeting. Using the target specific sequence as a molecular barcode is a particularly useful feature that enables highly specific analysis of the properties of targeting oligonucleotides.

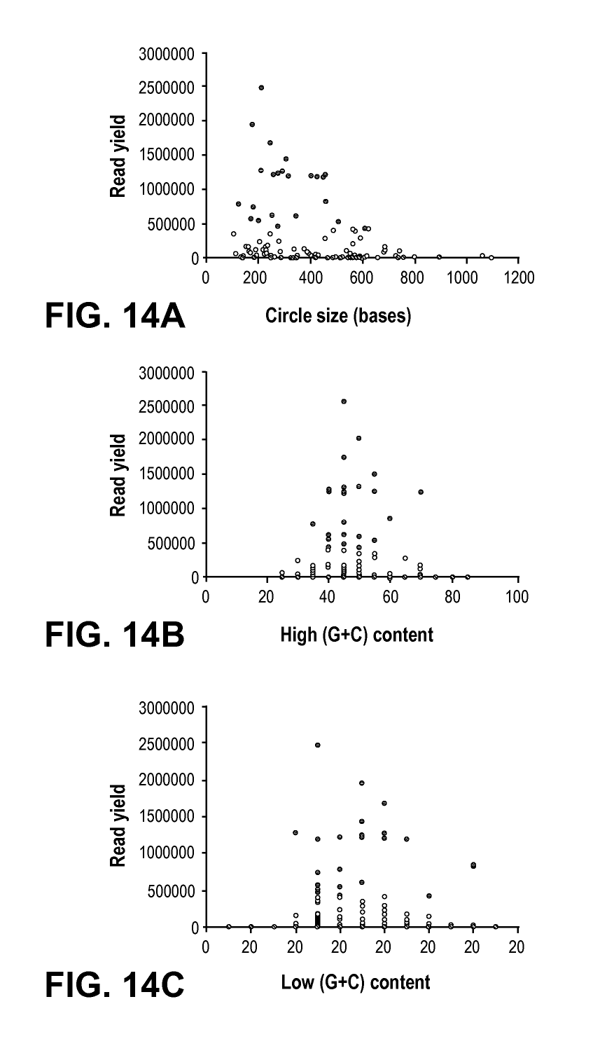

To investigate the capture properties of the assay we classified each targeting oligonucleotide based on their specific sequence yield from experiment 1. Out of 107 oligonucleotides, three categories were set up: 25 failed to generate targeted sequence, 25 were top performing and 57 performed moderately. We then evaluated properties of the capture oligonucleotides, such as guanine and cytosine (G+C) content of target specific 20-mers and size of the captured circle that were then linked with sequence yields (FIG. 14). The figure shows that circles between 150 and 600 bases perform robustly, while circles above 600 by fail or result in low capture yields (FIG. 14a). The low yields of the larger circles can be due to a combination of at least 3 factors: (1) larger circles may not form in the first place, (2) a PCR induced bias against larger circles at the amplificiation step, (3) reduced efficiency of cluster formation on the flowcell. Furthermore, it was determined that high (FIG. 14b) and low (G+C) (FIG. 14c) content of the target specific sites may be associated with lower yields or total failure of the oligonucleotides.

Simple optimization of the oligonucleotide design may improve the capture yields. For instance, the size of the circles should be restricted to 150-600 bases to comply with the Illumina sequencing system and (G+C) content of the 20-mer targeting sites should be normalized to 30-50% for more uniform coverage. We hypothesize that oligonucleotides with low (G+C) content do not properly anneal to targets during circularization. Conversely, high (G+C) represses DNA denature during heat shock and might affect the functionality of the oligonucleotides. These results suggest that properties of the targeting oligonucleotides that depend on circularization conditions, such as (G+C) content, should be normalized. Moreover, sizes of the captured fragments should comply with the sequencing system.

Genotyping Accuracy of Targeted Sequencing Library Preparation Method

To demonstrate the accuracy of our targeted resequencing assay, a genomic DNA sample (NA18507) of a Yuruban individual that has previously undergone whole genome sequencing was resequenced. The analysis was restricted to targeted regions with high fold-coverage (>30) sequencing data. Targeted resequencing of PCR amplified libraries was highly accurate as 99.4-99.8% of the targeted positions were concordant with the reference sequence (Table 1). Moreover, higher hybridization temperature during genomic circularization (see experiments 2-4) yielded better concordance (Table 1). Interestingly, amplification-free sequencing resulted in zero false positive findings even though the sequencing fold-coverage was considerably lower than in PCR libraries. Also, even though the sequence-fold coverage of the direct sequencing experiment is approximately 1000-fold lower than the coverage observed for the amplified single read experiments (Experiments 2,3,4), the number of captured bases at coverage >30 is similar at 2-3 kb. Together these results suggest that stringent hybridization conditions and amplification-free sequencing of the targeted libraries improve genotyping and reduce the amount of PCR artifacts.

Described above is a novel strategy to prepare NGS libraries of targeted DNA content with a single circularization step. The method is based on genomic circularization, but instead of amplifying the circles using a pair of universal primers and ligating adapters to the amplified material, include the adapter sequences are included in the capture oligonucleotide mediating the circularization. Adapted genomic circles can be directly sequenced or PCR library can be generated using regular sample preparation primers. We have demonstrated the concept of integrated library preparation and target enrichment and showed that our assay effectively captures targeted genomic regions with good coverage and high specificity.

The interest towards end-sequencing approaches has been increasing in concert with sequencing read lengths. For methods that require molecular amplification, the advantage of having random sequencing start sites is that PCR duplicates can be easily resolved by filtering reads derived from identical fragments. While high specificity of restriction endonucleases can be useful in variety of applications, it reduces the representation of the genomic complexity. The applicability of end-sequencing methods for DNA with reduced complexity has been limited, since restriction digestion fragments are inherently identical and the effects of molecular bottlenecking are indistinguishable. However, in single molecule applications such as the one presented here, every sequenced molecule is unique and filtering of duplicate fragments becomes obsolete. If sequencing read length continues to grow with current pace, it is not far in the future when entire restriction digested DNA fragments can be analyzed using intersecting paired-end reads.

Although the feasibility of the method has been demonstrated using the Illumina NGS system, the approach is generally applicable for generating sequencing libraries for different sequencing platforms. For example, the 454 (Roche) and the SOLiD (Applied Biosystems) platforms rely on preparing recombinant DNA sequencing libraries that have specific adaptor sequences at 3′ and 5′ ends and the PacBio RS system utilizes circular DNA as a template for sequencing. This suggests that the targeted circularization assay presented here may be applicable for variety of NGS systems.

Targeted resequencing applications are expected to provide the foundation for clinical genomics and high-throughput genetic diagnostics and catalyze the paradigm shift from translational to personalized medicine. This rapid and amplification-free solution provides a powerful tool for targeted and high-throughput analysis of the genome.

TABLE 2
Oligonucleotide features

| | | | Target start | | LH | RH | RH | Amplicon | Target |
|---|---|---|---|---|---|---|---|---|---|
| No. | Type | c/s | site | LH start | end | start | end | length | gene |
| 1 | Splint | 14 | 104306673 | 981 | 1000 | 1198 | 1217 | 237 | FRAP1 |
| 2 | Splint | 14 | 104307077 | 960 | 979 | 1186 | 1205 | 246 | FRAP1 |
| 3 | Splint | 14 | 104308697 | 295 | 314 | 1171 | 1190 | 896 | FRAP1 |
| 4 | Splint | 14 | 104309210 | 1000 | 1019 | 1496 | 1515 | 516 | FRAP1 |
| 5 | Splint | 14 | 104310244 | 1020 | 1039 | 1596 | 1615 | 596 | FRAP1 |
| 6 | Splint | 14 | 104311270 | 592 | 611 | 1333 | 1352 | 761 | TGFBR2 |
| 7 | Splint | 3 | 30622330 | 1000 | 1019 | 1875 | 1894 | 895 | EGFR |
| 8 | Splint | 3 | 30703830 | 1000 | 1019 | 1241 | 1260 | 261 | EGFR |
| 9 | Splint | 3 | 30706866 | 931 | 950 | 1263 | 1282 | 352 | EGFR |
| 10 | Splint | 1 | 11094446 | 798 | 817 | 1350 | 1369 | 572 | EGFR |
| 11 | Splint | 1 | 11095912 | 819 | 838 | 1219 | 1238 | 420 | MARK3 |
| 12 | Splint | 1 | 11096407 | 1000 | 1019 | 1206 | 1225 | 226 | MARK3 |
| 13 | Splint | 1 | 11096990 | 972 | 991 | 1156 | 1175 | 204 | MARK3 |
| 14 | Splint | 1 | 11102840 | 862 | 881 | 1186 | 1205 | 344 | AKT1 |
| 15 | Splint | 1 | 11103573 | 920 | 939 | 1231 | 1250 | 331 | AKT1 |
| 16 | Splint | 1 | 11109598 | 678 | 697 | 1222 | 1241 | 564 | AKT1 |
| 17 | Splint | 1 | 11110048 | 828 | 847 | 1212 | 1231 | 404 | TP53 |
| 18 | Splint | 1 | 11110449 | 951 | 970 | 1540 | 1559 | 609 | TP53 |
| 19 | Splint | 1 | 11114674 | 874 | 893 | 1339 | 1358 | 485 | TP53 |
| 20 | Splint | 1 | 11115945 | 762 | 781 | 1199 | 1218 | 457 | TP53 |
| 21 | Splint | 1 | 11126242 | 878 | 897 | 1201 | 1220 | 343 | TP53 |
| 22 | Splint | 1 | 11128270 | 530 | 549 | 1199 | 1218 | 689 | SMAD4 |
| 23 | Splint | 1 | 11138746 | 1000 | 1019 | 1229 | 1248 | 249 | AKT2 |
| 24 | Splint | 1 | 11186155 | 953 | 972 | 1226 | 1245 | 293 | AKT2 |
| 25 | Splint | 1 | 11190906 | 986 | 1005 | 1247 | 1266 | 281 | AKT2 |
| 26 | Splint | 1 | 11192408 | 724 | 743 | 1329 | 1348 | 625 | FRAP1 |
| 27 | Splint | 1 | 11193906 | 779 | 798 | 1269 | 1288 | 510 | FRAP1 |
| 28 | Splint | 1 | 11212519 | 666 | 685 | 1334 | 1353 | 688 | FRAP1 |
| 29 | Splint | 1 | 11214030 | 653 | 672 | 1176 | 1195 | 543 | FRAP1 |
| 30 | Splint | 1 | 11215737 | 893 | 912 | 1434 | 1453 | 561 | FRAP1 |
| 31 | Splint | 1 | 11219437 | 1000 | 1019 | 1405 | 1424 | 425 | FRAP1 |
| 32 | Splint | 1 | 11221897 | 1000 | 1019 | 1552 | 1571 | 572 | FRAP1 |
| 33 | Splint | 1 | 11237586 | 1000 | 1019 | 1397 | 1416 | 417 | FRAP1 |
| 34 | Splint | 1 | 11238527 | 963 | 982 | 1316 | 1335 | 373 | FRAP1 |
| 35 | Splint | 1 | 11240079 | 954 | 973 | 1329 | 1348 | 395 | FRAP1 |
| 36 | Splint | 14 | 102940116 | 955 | 974 | 1325 | 1344 | 390 | FRAP1 |
| 37 | Splint | 14 | 102997445 | 1002 | 1021 | 1194 | 1213 | 212 | FRAP1 |
| 38 | Splint | 14 | 103001383 | 925 | 944 | 1230 | 1249 | 325 | FRAP1 |
| 39 | Splint | 14 | 103002119 | 1000 | 1019 | 1309 | 1328 | 329 | FRAP1 |
| 40 | Splint | 14 | 103003073 | 988 | 1007 | 1559 | 1578 | 591 | FRAP1 |
| 41 | Splint | 19 | 45430569 | 1020 | 1039 | 1488 | 1507 | 488 | FRAP1 |
| 42 | Splint | 19 | 45431742 | 987 | 1006 | 1429 | 1448 | 462 | FRAP1 |
| 43 | Splint | 19 | 45431960 | 769 | 788 | 1211 | 1230 | 462 | FRAP1 |
| 44 | Splint | 19 | 45432954 | 1000 | 1019 | 1500 | 1519 | 520 | FRAP1 |
| 45 | Splint | 19 | 45434666 | 1000 | 1019 | 1640 | 1659 | 660 | FRAP1 |
| 46 | Splint | 19 | 45435602 | 865 | 884 | 1273 | 1292 | 428 | TGFBR2 |
| 47 | Splint | 19 | 45436742 | 602 | 621 | 1149 | 1168 | 567 | TGFBR2 |
| 48 | Splint | 19 | 45438635 | 631 | 650 | 1228 | 1247 | 617 | TGFBR2 |
| 49 | Splint | 19 | 45439231 | 652 | 671 | 1217 | 1236 | 585 | TGFBR2 |
| 50 | Splint | 19 | 45451855 | 131 | 150 | 1175 | 1194 | 1064 | APC |
| 51 | Splint | 17 | 7512602 | 827 | 846 | 1145 | 1164 | 338 | APC |
| 52 | Splint | 17 | 7516528 | 861 | 880 | 1399 | 1418 | 558 | APC |
| 53 | Splint | 17 | 7517174 | 1000 | 1019 | 1566 | 1585 | 586 | APC |
| 54 | Splint | 17 | 7518987 | 914 | 933 | 1362 | 1381 | 468 | APC |
| 55 | Splint | 17 | 7519375 | 526 | 545 | 1085 | 1104 | 579 | APC |
| 56 | Splint | 17 | 7519514 | 1040 | 1059 | 1758 | 1777 | 738 | APC |
| 57 | Splint | 7 | 55177442 | 752 | 771 | 1416 | 1435 | 684 | APC |
| 58 | Splint | 7 | 55185431 | 975 | 994 | 1272 | 1291 | 317 | APC |
| 59 | Splint | 7 | 55186683 | 863 | 882 | 1416 | 1435 | 573 | EGFR |
| 60 | Splint | 7 | 55188148 | 730 | 749 | 1225 | 1244 | 515 | EGFR |
| 61 | Splint | 7 | 55189967 | 926 | 945 | 1246 | 1265 | 340 | EGFR |
| 62 | Splint | 7 | 55191800 | 671 | 690 | 1186 | 1205 | 535 | EGFR |
| 63 | Splint | 7 | 55194276 | 882 | 901 | 1320 | 1339 | 458 | EGFR |
| 64 | Splint | 7 | 55197870 | 901 | 920 | 1379 | 1398 | 498 | EGFR |
| 65 | Splint | 7 | 55205312 | 982 | 1001 | 1102 | 1121 | 140 | EGFR |
| 66 | Splint | 7 | 55208058 | 833 | 852 | 1556 | 1575 | 743 | EGFR |
| 67 | Splint | 7 | 55215430 | 678 | 697 | 1269 | 1288 | 611 | EGFR |
| 68 | Splint | 7 | 55225856 | 859 | 878 | 1266 | 1285 | 427 | KRAS |
| 69 | Splint | 7 | 55226903 | 990 | 1009 | 1171 | 1190 | 201 | MARK3 |
| 70 | Splint | 7 | 55232854 | 755 | 774 | 1287 | 1306 | 552 | MARK3 |
| 71 | Splint | 7 | 55234453 | 984 | 1003 | 1243 | 1262 | 279 | AKT1 |
| 72 | Splint | 7 | 55235325 | 870 | 889 | 1251 | 1270 | 401 | AKT1 |
| 73 | Splint | 7 | 55235872 | 944 | 963 | 1111 | 1130 | 187 | AKT1 |
| 74 | Splint | 7 | 55236654 | 723 | 742 | 1172 | 1191 | 469 | AKT1 |
| 75 | Splint | 14 | 104309583 | 1001 | 1020 | 1123 | 1142 | 142 | AKT1 |
| 76 | Splint | 14 | 104309583 | 1145 | 1164 | 1412 | 1431 | 287 | TP53 |
| 77 | Splint | 3 | 30665716 | 1021 | 1040 | 1238 | 1257 | 237 | SMAD4 |
| 78 | Splint | 3 | 30687084 | 1001 | 1020 | 1149 | 1168 | 168 | AKT2 |
| 79 | Splint | 3 | 30687084 | 1171 | 1190 | 1882 | 1901 | 731 | AKT2 |
| 80 | Splint | 12 | 25268765 | 1001 | 1020 | 1171 | 1190 | 190 | AKT2 |
| 81 | Splint | 5 | 112117437 | 1081 | 1100 | 1187 | 1206 | 126 | AKT2 |
| 82 | Splint | 5 | 112184442 | 1001 | 1020 | 1146 | 1165 | 165 | AKT2 |
| 83 | Splint | 5 | 112200099 | 1100 | 1119 | 1251 | 1270 | 171 | FRAP1 |
| 84 | Splint | 5 | 112200099 | 1271 | 1290 | 1410 | 1429 | 159 | FRAP1 |
| 85 | Splint | 5 | 112200099 | 1430 | 1449 | 1516 | 1535 | 106 | FRAP1 |
| 86 | Splint | 5 | 112200099 | 1536 | 1555 | 1965 | 1984 | 449 | FRAP1 |
| 87 | Splint | 5 | 112200099 | 1985 | 2004 | 2161 | 2180 | 196 | FRAP1 |
| 88 | Splint | 5 | 112200099 | 2181 | 2200 | 2417 | 2436 | 256 | TGFBR2 |
| 89 | Splint | 5 | 112200099 | 2457 | 2476 | 2616 | 2635 | 179 | APC |
| 90 | Splint | 5 | 112200099 | 2636 | 2655 | 2836 | 2855 | 220 | APC |
| 91 | Splint | 5 | 112200099 | 2856 | 2875 | 3639 | 3658 | 803 | APC |
| 92 | Splint | 5 | 112200099 | 3659 | 3678 | 4258 | 4277 | 619 | APC |
| 93 | Splint | 5 | 112200099 | 4278 | 4297 | 4470 | 4489 | 212 | APC |
| 94 | Splint | 5 | 112200099 | 4490 | 4509 | 4716 | 4735 | 246 | APC |
| 95 | Splint | 5 | 112200099 | 4754 | 4773 | 5831 | 5850 | 1097 | APC |
| 96 | Splint | 5 | 112200099 | 6044 | 6063 | 6256 | 6275 | 232 | APC |
| 97 | Splint | 5 | 112200099 | 6296 | 6315 | 6429 | 6448 | 153 | APC |
| 98 | Splint | 5 | 112200099 | 7176 | 7195 | 7426 | 7445 | 270 | APC |
| 99 | Splint | 5 | 112200099 | 7446 | 7465 | 7604 | 7623 | 178 | EGFR |
| 100 | Splint | 1 | 11210262 | 1088 | 1107 | 1333 | 1352 | 265 | EGFR |
| 101 | Splint | 1 | 11214992 | 1001 | 1020 | 1115 | 1134 | 134 | EGFR |
| 102 | Splint | 1 | 11219996 | 1016 | 1035 | 1278 | 1297 | 282 | EGFR |
| 103 | Splint | 1 | 11240842 | 1001 | 1020 | 1227 | 1246 | 246 | EGFR |
| 104 | Splint | 18 | 46828004 | 1001 | 1020 | 1117 | 1136 | 136 | MARK3 |
| 105 | Splint | 18 | 46828004 | 1165 | 1184 | 1257 | 1276 | 112 | MARK3 |
| 106 | Splint | 14 | 103026817 | 1001 | 1020 | 1267 | 1286 | 286 | AKT2 |
| 107 | Splint | 14 | 103037922 | 1023 | 1042 | 1306 | 1325 | 303 | AKT2 |
| 108 | Vector | NA | NA | NA | NA | NA | NA | NA | NA |

What is claimed is:

1. A method of sequencing comprising:
a) digesting a sample comprising genomic DNA using a restriction enzyme to produce a digested sample;
b) producing a circular nucleic acid comprising i. a splint oligonucleotide, ii. a vector oligonucleotide comprises a binding site for a first sequencing primer iii. a target genomic fragment, and iv. a duplex region in which the 5′ end of said vector oligonucleotide is ligatably adjacent to the 3′ end of the target genomic fragment, and the 3′ end of said vector oligonucleotide is ligatably adjacent to the 5′ end of said target genomic fragment by:
contacting, under hybridization conditions, said digested sample with:
i. said vector oligonucleotide; and
ii. said splint oligonucleotide, wherein said splint oligonucleotide comprises:
a central region that hybridizes to the entirety of said vector oligonucleotide;
a 5′ region that hybridizes to a first region in a target genomic fragment in said digested sample, and
a 3′ region that hybridizes to a second region in said target genomic fragment;
and, optionally enzymatic treatment remove any 5′ overhang from said target genomic fragment to make the 3′ end of said vector oligonucleotide ligatably adjacent to the 5′ end of said target genomic fragment;
c) contacting said circular nucleic acid with a ligase, thereby ligating the 5′ end of said vector oligonucleotide to the 3′ end of the target genomic fragment and ligating the 3′ end of said vector oligonucleotide to the 5′ end of the target genomic fragment to produce a circular DNA molecule;
d) separating said circular DNA molecule from said splint oligonucleotide; and
e) sequencing the target genomic fragment of said circular DNA molecule using said first sequencing primer.

2. The method of claim 1, wherein said vector oligonucleotide further comprises a second binding site for a second sequencing primer and said sequencing step e) comprises sequencing the target genomic fragment of said circular DNA molecule using said first and second sequencing primers.

3. The method of claim 1, further comprising, prior to said sequencing set e), amplifying the target genomic fragment of said circular DNA molecule by polymerase chain reaction (PCR) using a pair of primers that bind to primer sites that are also present in said vector oligonucleotide in addition to said sequencing primer site.

4. The method of claim 1, further comprising linearizing the circular DNA molecule prior to said sequencing step e).

5. The method of claim 1, wherein said contacting steps b) and c) are done in single vessel without the addition of further reagents.

6. The method of claim 1, wherein steps d) and e) are done in the absence of amplifying said circular DNA.

7. The method of claim 1, wherein step b) comprises enzymatic treatment to remove any 5′ overhang from said target genomic fragment to make the 3′ end of said vector oligonucleotide ligatably adjacent to the 5′ end of said target genomic fragment.

8. The method of claim 7, wherein said enzymatic treatment comprises contacting with a FLAP endonuclease.

9. The method of claim 8, wherein said FLAP endonuclease is Taq.

10. The method of claim 5, wherein said contacting steps b) and c) are done in a single vessel in which said genomic fragment, said vector oligonucleotide, said splint oligonucleotide and a thermostable ligase are thermally cycled through multiple rounds of a temperature suitable for denaturation and a temperature suitable for hybridization and ligation.

11. The method of claim 3, wherein said amplifying is clonal amplification in which said circular DNA molecules are amplified in separate reactions that are spatially distinct from one another.

12. The method of claim 11, wherein said clonal amplification is done by bridge PCR.

13. The method of claim 11, wherein said clonal amplification is done by emulsion PCR.

14. The method of claim 3, wherein said amplifying is a bulk amplification in which said circular DNA molecules are amplified in a single reaction containing a plurality of said circular DNA molecules.

15. The method of claim 1, wherein said method isolates and provides the nucleotide sequence of known loci of a genome.

16. The method of claim 1, wherein said method isolates and provides the nucleotide sequence of a partitioned genome.

17. The method of claim 1, wherein said sequencing is done by sequencing is by a next generation sequencing method.

18. A kit comprising:
i. a vector oligonucleotide comprising a first binding site for a sequencing primer and a second binding site for a second sequencing primer; and
ii. a splint oligonucleotide that hybridizes to said the vector oligonucleotide and to the nucleotide sequences at the ends of a plurality of restriction fragments in a mammalian genome,
wherein said vector and splint oligonucleotides are characterized in that, when hybridized with said restriction fragment, they produce a circular nucleic acid comprising a duplex region in which at least the 5′ end of said vector oligonucleotide is ligatably adjacent to the 3′ end of the genomic fragment.

19. The kit of claim 18, further comprising a ligase.

20. The kit of claim 18, further comprising primers that bind to sites in said vector oligonucleotide and that can amplify said genomic fragments, once ligated to said vector oligonucleotide.


SEQUENCE LISTING (108 sequences)

SEQ ID NO: 1 - DNA, 159 bp, Artificial Sequence
  Synthetic oligonucleotide
agtaggaagccaaccucttaagaucggaagagcggtucagcaggaatgccgagaccgauctcgtatgccgtcutctgcttgaatgauacggcgaccaccgagauctacactctutccctacacgacgcucttccgatctaatacagatcauggcacgag

SEQ ID NO: 2 - DNA, 159 bp, Artificial Sequence
  Synthetic oligonucleotide
cttcgctccccacucccagcagaucggaagagcggtucagcaggaatgccgagaccgauctcgtatgccgtcutctgcttgaatgauacggcgaccaccgagauctacactctutccctacacgacgcucttccgatctaaacatcugaagccaaaaaa

SEQ ID NO: 3 - DNA, 159 bp, Artificial Sequence
  Synthetic oligonucleotide
accgccaccugcccaggcccagaucggaagagcggtucagcaggaatgccgagaccgauctcgtatgccgtcutctgcttgaatgauacggcgaccaccgagauctacactctutccctacacgacgcucttccgatctatgggagaacaggugcccag

SEQ ID NO: 4 - DNA, 159 bp, Artificial Sequence
  Synthetic oligonucleotide
acctccaucccctcatccccagaucggaagagcggtucagcaggaatgccgagaccgauctcgtatgccgtcutctgcttgaatgauacggcgaccaccgagauctacactctutccctacacgacgcucttccgatctagatcacagacutcgggctg

SEQ ID NO: 5 - DNA, 159 bp, Artificial Sequence
  Synthetic oligonucleotide
ggctgcgggggauggacttcagaucggaagagcggtucagcaggaatgccgagaccgauctcgtatgccgtcutctgcttgaatgauacggcgaccaccgagauctacactctutccctacacgacgcucttccgatctaaagcacagucgcctgtggt

SEQ ID NO: 6 - DNA, 159 bp, Artificial Sequence
  Synthetic oligonucleotide
accagggucagcaagcggcgagaucggaagagcggtucagcaggaatgccgagaccgauctcgtatgccgtcutctgcttgaatgauacggcgaccaccgagauctacactctutccctacacgacgcucttccgatctaagcaaaggccccauctgca

SEQ ID NO: 7 - DNA, 159 bp, Artificial Sequence
  Synthetic oligonucleotide
ccccagcgcagcggacggcgagaucggaagagcggtucagcaggaatgccgagaccgauctcgtatgccgtcutctgcttgaatgauacggcgaccaccgagauctacactctutccctacacgacgcucttccgatctacccttcgguttccaacaaa

SEQ ID NO: 8 - DNA, 159 bp, Artificial Sequence
  Synthetic oligonucleotide
tgagaatggcaugtgcagccagaucggaagagcggtucagcaggaatgccgagaccgauctcgtatgccgtcutctgcttgaatgauacggcgaccaccgagauctacactctutccctacacgacgcucttccgatctagctcaatautccagaattc

SEQ ID NO: 9 - DNA, 159 bp, Artificial Sequence
  Synthetic oligonucleotide
aggctgccccuctcaccaaaagaucggaagagcggtucagcaggaatgccgagaccgauctcgtatgccgtcutctgcttgaatgauacggcgaccaccgagauctacactctutccctacacgacgcucttccgatctaatgcacaggcacutttgga

SEQ ID NO: 10 - DNA, 159 bp, Artificial Sequence
  Synthetic oligonucleotide
taaggtcaaaguatgatttaagaucggaagagcggtucagcaggaatgccgagaccgauctcgtatgccgtcutctgcttgaatgauacggcgaccaccgagauctacactctutccctacacgacgcucttccgatctagttatgccucctactgtca

SEQ ID NO: 11 - DNA, 159 bp, Artificial Sequence
  Synthetic oligonucleotide
gttactaacuctccacccaaagaucggaagagcggtucagcaggaatgccgagaccgauctcgtatgccgtcutctgcttgaatgauacggcgaccaccgagauctacactctutccctacacgacgcucttccgatctaacctctttccuttataaat

SEQ ID NO: 12 - DNA, 159 bp, Artificial Sequence
  Synthetic oligonucleotide
aatgggaucaggacagttacagaucggaagagcggtucagcaggaatgccgagaccgauctcgtatgccgtcutctgcttgaatgauacggcgaccaccgagauctacactctutccctacacgacgcucttccgatctaaatcagugcaggtgatgca

SEQ ID NO: 13 - DNA, 159 bp, Artificial Sequence
  Synthetic oligonucleotide
tcaacagagauaacggatgaagaucggaagagcggtucagcaggaatgccgagaccgauctcgtatgccgtcutctgcttgaatgauacggcgaccaccgagauctacactctutccctacacgacgcucttccgatctagtctgccuacagtgtcaga

SEQ ID NO: 14 - DNA, 159 bp, Artificial Sequence
  Synthetic oligonucleotide
acaactgttcaguaagagagagaucggaagagcggtucagcaggaatgccgagaccgauctcgtatgccgtcutctgcttgaatgauacggcgaccaccgagauctacactctutccctacacgacgcucttccgatctaactactcautctaactctg

SEQ ID NO: 15 - DNA, 159 bp, Artificial Sequence
  Synthetic oligonucleotide
cgtaaagagaguatacccttagaucggaagagcggtucagcaggaatgccgagaccgauctcgtatgccgtcutctgcttgaatgauacggcgaccaccgagauctacactctutccctacacgacgcucttccgatctaaccttctucaaagctgatt

SEQ ID NO: 16 - DNA, 159 bp, Artificial Sequence
  Synthetic oligonucleotide
aagagctgguatgaattttaagaucggaagagcggtucagcaggaatgccgagaccgauctcgtatgccgtcutctgcttgaatgauacggcgaccaccgagauctacactctutccctacacgacgcucttccgatctaaggatgttuccttcagagt

SEQ ID NO: 17 - DNA, 159 bp, Artificial Sequence
  Synthetic oligonucleotide
ggcaaagaucaattcttttaagaucggaagagcggtucagcaggaatgccgagaccgauctcgtatgccgtcutctgcttgaatgauacggcgaccaccgagauctacactctutccctacacgacgcucttccgatctacatctgctugagactacca

SEQ ID NO: 18 - DNA, 159 bp, Artificial Sequence
  Synthetic oligonucleotide
cacctttacccuctgggttaagaucggaagagcggtucagcaggaatgccgagaccgauctcgtatgccgtcutctgcttgaatgauacggcgaccaccgagauctacactctutccctacacgacgcucttccgatctacacacacugccttgtgaca

SEQ ID NO: 19 - DNA, 159 bp, Artificial Sequence
  Synthetic oligonucleotide
gcttttggaauacatttttaagaucggaagagcggtucagcaggaatgccgagaccgauctcgtatgccgtcutctgcttgaatgauacggcgaccaccgagauctacactctutccctacacgacgcucttccgatctaacttguagggggaaatgca

SEQ ID NO: 20 - DNA, 159 bp, Artificial Sequence
  Synthetic oligonucleotide
ggcttguggcccagcttcagagaucggaagagcggtucagcaggaatgccgagaccgauctcgtatgccgtcutctgcttgaatgauacggcgaccaccgagauctacactctutccctacacgacgcucttccgatctaaaaaaaaaaagggaggggc

SEQ ID NO: 21 - DNA, 159 bp, Artificial Sequence
  Synthetic oligonucleotide
caauccaaaagacaggautaagaucggaagagcggtucagcaggaatgccgagaccgauctcgtatgccgtcutctgcttgaatgauacggcgaccaccgagauctacactctutccctacacgacgcucttccgatctagtttaggugaggagccttt

SEQ ID NO: 22 - DNA, 159 bp, Artificial Sequence
  Synthetic oligonucleotide
gggggtuggctggttgggctagaucggaagagcggtucagcaggaatgccgagaccgauctcgtatgccgtcutctgcttgaatgauacggcgaccaccgagauctacactctutccctacacgacgcucttccgatctagtgctcagaucatttaccc

SEQ ID NO: 23 - DNA, 159 bp, Artificial Sequence
  Synthetic oligonucleotide
tgcaagagacutcgtctcttagaucggaagagcggtucagcaggaatgccgagaccgauctcgtatgccgtcutctgcttgaatgauacggcgaccaccgagauctacactctutccctacacgacgcucttccgatctaacaatgtgaucccttcttc

SEQ ID NO: 24 - DNA, 159 bp, Artificial Sequence
  Synthetic oligonucleotide
cagttucaagggccaattgaagaucggaagagcggtucagcaggaatgccgagaccgauctcgtatgccgtcutctgcttgaatgauacggcgaccaccgagauctacactctutccctacacgacgcucttccgatctaaaataccagccccutgatt

SEQ ID NO: 25 - DNA, 159 bp, Artificial Sequence
  Synthetic oligonucleotide
tggtttaggaaauatccttaagaucggaagagcggtucagcaggaatgccgagaccgauctcgtatgccgtcutctgcttgaatgauacggcgaccaccgagauctacactctutccctacacgacgcucttccgatctaaagttttgaguaagtgaga

SEQ ID NO: 26 - DNA, 159 bp, Artificial Sequence
  Synthetic oligonucleotide
tttatgatgguggcctcttaagaucggaagagcggtucagcaggaatgccgagaccgauctcgtatgccgtcutctgcttgaatgauacggcgaccaccgagauctacactctutccctacacgacgcucttccgatctaatggtuggggggaaaaaaa

SEQ ID NO: 27 - DNA, 159 bp, Artificial Sequence
  Synthetic oligonucleotide
cgtgactgagggugagcttaagaucggaagagcggtucagcaggaatgccgagaccgauctcgtatgccgtcutctgcttgaatgauacggcgaccaccgagauctacactctutccctacacgacgcucttccgatctagaaacaaacugtttatttg

SEQ ID NO: 28 - DNA, 159 bp, Artificial Sequence
  Synthetic oligonucleotide
gacactgucagagcatgttaagaucggaagagcggtucagcaggaatgccgagaccgauctcgtatgccgtcutctgcttgaatgauacggcgaccaccgagauctacactctutccctacacgacgcucttccgatctaaacaggaagaaucaattct

SEQ ID NO: 29 - DNA, 159 bp, Artificial Sequence
  Synthetic oligonucleotide
ttagaaattcuggcattagaagaucggaagagcggtucagcaggaatgccgagaccgauctcgtatgccgtcutctgcttgaatgauacggcgaccaccgagauctacactctutccctacacgacgcucttccgatctattttagtcuagtcatctaa

SEQ ID NO: 30 - DNA, 159 bp, Artificial Sequence
  Synthetic oligonucleotide
tctctaaagcagagcutttaagaucggaagagcggtucagcaggaatgccgagaccgauctcgtatgccgtcutctgcttgaatgauacggcgaccaccgagauctacactctutccctacacgacgcucttccgatctactgaaagtgcutgatatgg

SEQ ID NO: 31 - DNA, 159 bp, Artificial Sequence
  Synthetic oligonucleotide
gggaagacaggacuctcgctagaucggaagagcggtucagcaggaatgccgagaccgauctcgtatgccgtcutctgcttgaatgauacggcgaccaccgagauctacactctutccctacacgacgcucttccgatctattctttugtcttttactct

SEQ ID NO: 32 - DNA, 159 bp, Artificial Sequence
  Synthetic oligonucleotide
gtgagagaugctggaaacttagaucggaagagcggtucagcaggaatgccgagaccgauctcgtatgccgtcutctgcttgaatgauacggcgaccaccgagauctacactctutccctacacgacgcucttccgatctatctgctucccagagtgttt

SEQ ID NO: 33 - DNA, 159 bp, Artificial Sequence
  Synthetic oligonucleotide
aaagcacatcugcgtagagaagaucggaagagcggtucagcaggaatgccgagaccgauctcgtatgccgtcutctgcttgaatgauacggcgaccaccgagauctacactctutccctacacgacgcucttccgatctatcaatttugctttccttcc

SEQ ID NO: 34 - DNA, 159 bp, Artificial Sequence
  Synthetic oligonucleotide
ggggcgtaugctggccaggaagaucggaagagcggtucagcaggaatgccgagaccgauctcgtatgccgtcutctgcttgaatgauacggcgaccaccgagauctacactctutccctacacgacgcucttccgatctaggaatgagccucagaagga

SEQ ID NO: 35 - DNA, 159 bp, Artificial Sequence
  Synthetic oligonucleotide
tgcaatcatcugattatttaagaucggaagagcggtucagcaggaatgccgagaccgauctcgtatgccgtcutctgcttgaatgauacggcgaccaccgagauctacactctutccctacacgacgcucttccgatctagcaaataugaactagtatc

SEQ ID NO: 36 - DNA, 159 bp, Artificial Sequence
  Synthetic oligonucleotide
ggccttatttcttttutttaagaucggaagagcggtucagcaggaatgccgagaccgauctcgtatgccgtcutctgcttgaatgauacggcgaccaccgagauctacactctutccctacacgacgcucttccgatctaagtaggaaugttttttctt

SEQ ID NO: 37 - DNA, 159 bp, Artificial Sequence
  Synthetic oligonucleotide
agccttattagcatttuttaagaucggaagagcggtucagcaggaatgccgagaccgauctcgtatgccgtcutctgcttgaatgauacggcgaccaccgagauctacactctutccctacacgacgcucttccgatctattacgaauctgcaccagtg

SEQ ID NO: 38 - DNA, 159 bp, Artificial Sequence
  Synthetic oligonucleotide
atgtgaataauggaaagttaagaucggaagagcggtucagcaggaatgccgagaccgauctcgtatgccgtcutctgcttgaatgauacggcgaccaccgagauctacactctutccctacacgacgcucttccgatctaaaaatatuttttgagttta

SEQ ID NO: 39 - DNA, 159 bp, Artificial Sequence
  Synthetic oligonucleotide
tttgttugggtaagaaatcaagaucggaagagcggtucagcaggaatgccgagaccgauctcgtatgccgtcutctgcttgaatgauacggcgaccaccgagauctacactctutccctacacgacgcucttccgatctaaatagcutataatagtcat

SEQ ID NO: 40 - DNA, 159 bp, Artificial Sequence
  Synthetic oligonucleotide
gtcttcagcucctggccttaagaucggaagagcggtucagcaggaatgccgagaccgauctcgtatgccgtcutctgcttgaatgauacggcgaccaccgagauctacactctutccctacacgacgcucttccgatctacttcctaauaagccatttg

SEQ ID NO: 41 - DNA, 159 bp, Artificial Sequence
  Synthetic oligonucleotide
acgcagaggacgcacgcucgagaucggaagagcggtucagcaggaatgccgagaccgauctcgtatgccgtcutctgcttgaatgauacggcgaccaccgagauctacactctutccctacacgacgcucttccgatctaagcaggcctutagaaagcc

SEQ ID NO: 42 - DNA, 159 bp, Artificial Sequence
  Synthetic oligonucleotide
caggccctguatggcccttaagaucggaagagcggtucagcaggaatgccgagaccgauctcgtatgccgtcutctgcttgaatgauacggcgaccaccgagauctacactctutccctacacgacgcucttccgatctagagcagaucccatccctcc

SEQ ID NO: 43 - DNA, 159 bp, Artificial Sequence
  Synthetic oligonucleotide
caggcccugtatggcccttaagaucggaagagcggtucagcaggaatgccgagaccgauctcgtatgccgtcutctgcttgaatgauacggcgaccaccgagauctacactctutccctacacgacgcucttccgatctagagcagaucccatccctcc

SEQ ID NO: 44 - DNA, 159 bp, Artificial Sequence
  Synthetic oligonucleotide
gaggccaaggguagggggatagaucggaagagcggtucagcaggaatgccgagaccgauctcgtatgccgtcutctgcttgaatgauacggcgaccaccgagauctacactctutccctacacgacgcucttccgatctaaggacagaaaguctagaaa

SEQ ID NO: 45 - DNA, 159 bp, Artificial Sequence
  Synthetic oligonucleotide
gtggcaugcacaccacatggagaucggaagagcggtucagcaggaatgccgagaccgauctcgtatgccgtcutctgcttgaatgauacggcgaccaccgagauctacactctutccctacacgacgcucttccgatctaaaccuagcagccctccgtt

SEQ ID NO: 46 - DNA, 159 bp, Artificial Sequence
  Synthetic oligonucleotide
ctgcacctttuggggcattaagaucggaagagcggtucagcaggaatgccgagaccgauctcgtatgccgtcutctgcttgaatgauacggcgaccaccgagauctacactctutccctacacgacgcucttccgatctacctcuctgagcctcagtga

SEQ ID NO: 47 - DNA, 159 bp, Artificial Sequence
  Synthetic oligonucleotide
gcaacaccacaccugcccacagaucggaagagcggtucagcaggaatgccgagaccgauctcgtatgccgtcutctgcttgaatgauacggcgaccaccgagauctacactctutccctacacgacgcucttccgatctactgguggccactggccttg

SEQ ID NO: 48 - DNA, 159 bp, Artificial Sequence
  Synthetic oligonucleotide
gcccgtgggaggaaautttaagaucggaagagcggtucagcaggaatgccgagaccgauctcgtatgccgtcutctgcttgaatgauacggcgaccaccgagauctacactctutccctacacgacgcucttccgatctaattttugaatgtctttgga

SEQ ID NO: 49 - DNA, 159 bp, Artificial Sequence
  Synthetic oligonucleotide
acacgtgagucccagcagccagaucggaagagcggtucagcaggaatgccgagaccgauctcgtatgccgtcutctgcttgaatgauacggcgaccaccgagauctacactctutccctacacgacgcucttccgatcuacaaaagaaagaggaaaacc

SEQ ID NO: 50 - DNA, 159 bp, Artificial Sequence
  Synthetic oligonucleotide
ggaacagacagcagggggcuagaucggaagagcggtucagcaggaatgccgagaccgauctcgtatgccgtcutctgcttgaatgauacggcgaccaccgagauctacactctutccctacacgacgcucttccgatctagaatguaggtcctgccggg

SEQ ID NO: 51 - DNA, 159 bp, Artificial Sequence
  Synthetic oligonucleotide
gaagcagggaggagagaugaagaucggaagagcggtucagcaggaatgccgagaccgauctcgtatgccgtcutctgcttgaatgauacggcgaccaccgagauctacactctutccctacacgacgcucttccgatctaaatcuaagctggtatgtcc

SEQ ID NO: 52 - DNA, 159 bp, Artificial Sequence
  Synthetic oligonucleotide
ggtcctacctgtcccauttaagaucggaagagcggtucagcaggaatgccgagaccgauctcgtatgccgtcutctgcttgaatgauacggcgaccaccgagauctacactctutccctacacgacgcucttccgatctaaatatauattatggtataa

SEQ ID NO: 53 - DNA, 159 bp, Artificial Sequence
  Synthetic oligonucleotide
cctgctgugccccagcctctagaucggaagagcggtucagcaggaatgccgagaccgauctcgtatgccgtcutctgcttgaatgauacggcgaccaccgagauctacactctutccctacacgacgcucttccgatctactatugcacagttgaaaaa

SEQ ID NO: 54 - DNA, 159 bp, Artificial Sequence
  Synthetic oligonucleotide
caggtccucagccccccagcagaucggaagagcggtucagcaggaatgccgagaccgauctcgtatgccgtcutctgcttgaatgauacggcgaccaccgagauctacactctutccctacacgacgcucttccgatctagtgggtaaacuataaaaaa

SEQ ID NO: 55 - DNA, 159 bp, Artificial Sequence
  Synthetic oligonucleotide
agcagaaagtcagucccatgagaucggaagagcggtucagcaggaatgccgagaccgauctcgtatgccgtcutctgcttgaatgauacggcgaccaccgagauctacactctutccctacacgacgcucttccgatctagtggguaaactataaaaaa

SEQ ID NO: 56 - DNA, 159 bp, Artificial Sequence
  Synthetic oligonucleotide
atggaaactgtgaguggatcagaucggaagagcggtucagcaggaatgccgagaccgauctcgtatgccgtcutctgcttgaatgauacggcgaccaccgagauctacactctutccctacacgacgcucttccgatctacaggaggugggagcagggc

SEQ ID NO: 57 - DNA, 159 bp, Artificial Sequence
  Synthetic oligonucleotide
tactggaaugggaagatttaagaucggaagagcggtucagcaggaatgccgagaccgauctcgtatgccgtcutctgcttgaatgauacggcgaccaccgagauctacactctutccctacacgacgcucttccgatctatttccatuttcactggaga

SEQ ID NO: 58 - DNA, 159 bp, Artificial Sequence
  Synthetic oligonucleotide
aatctcaccgcaugcagttaagaucggaagagcggtucagcaggaatgccgagaccgauctcgtatgccgtcutctgcttgaatgauacggcgaccaccgagauctacactctutccctacacgacgcucttccgatctacucaggcccgggaaagggc

SEQ ID NO: 59 - DNA, 159 bp, Artificial Sequence
  Synthetic oligonucleotide
ctctcccagutgaatgcttaagaucggaagagcggtucagcaggaatgccgagaccgauctcgtatgccgtcutctgcttgaatgauacggcgaccaccgagauctacactctutccctacacgacgcucttccgatctaaaataaaucaggagaaaaa

SEQ ID NO: 60 - DNA, 159 bp, Artificial Sequence
  Synthetic oligonucleotide
caggcatcctugtcccgctcagaucggaagagcggtucagcaggaatgccgagaccgauctcgtatgccgtcutctgcttgaatgauacggcgaccaccgagauctacactctutccctacacgacgcucttccgatctaacataatutcattccatag

SEQ ID NO: 61 - DNA, 159 bp, Artificial Sequence
  Synthetic oligonucleotide
acatcttccucttctcattaagaucggaagagcggtucagcaggaatgccgagaccgauctcgtatgccgtcutctgcttgaatgauacggcgaccaccgagauctacactctutccctacacgacgcucttccgatctaagtaaauaaagccaaagga

SEQ ID NO: 62 - DNA, 159 bp, Artificial Sequence
  Synthetic oligonucleotide
ttagttggaaautaggcttaagaucggaagagcggtucagcaggaatgccgagaccgauctcgtatgccgtcutctgcttgaatgauacggcgaccaccgagauctacactctutccctacacgacgcucttccgatctaggttggaaucaaataagga

SEQ ID NO: 63 - DNA, 159 bp, Artificial Sequence
  Synthetic oligonucleotide
tgaagggcuattcccatttaagaucggaagagcggtucagcaggaatgccgagaccgauctcgtatgccgtcutctgcttgaatgauacggcgaccaccgagauctacactctutccctacacgacgcucttccgatctaaaacaccugcagttttcaa

SEQ ID NO: 64 - DNA, 159 bp, Artificial Sequence
  Synthetic oligonucleotide
ttatcaaatccucacatttaagaucggaagagcggtucagcaggaatgccgagaccgauctcgtatgccgtcutctgcttgaatgauacggcgaccaccgagauctacactctutccctacacgacgcucttccgatctacaaaaucagctgattatat

SEQ ID NO: 65 - DNA, 159 bp, Artificial Sequence
  Synthetic oligonucleotide
agctctgtgucacatggaccagaucggaagagcggtucagcaggaatgccgagaccgauctcgtatgccgtcutctgcttgaatgauacggcgaccaccgagauctacactctutccctacacgacgcucttccgatctaaaatcuccaaaatatatgc

SEQ ID NO: 66 - DNA, 159 bp, Artificial Sequence
  Synthetic oligonucleotide
gcctagacgcagcaucattaagaucggaagagcggtucagcaggaatgccgagaccgauctcgtatgccgtcutctgcttgaatgauacggcgaccaccgagauctacactctutccctacacgacgcucttccgatctaagaacaccugtatcagagc

SEQ ID NO: 67 - DNA, 159 bp, Artificial Sequence
  Synthetic oligonucleotide
ataaggagccaggaucctcaagaucggaagagcggtucagcaggaatgccgagaccgauctcgtatgccgtcutctgcttgaatgauacggcgaccaccgagauctacactctutccctacacgacgcucttccgatctaaacacagcaucctcaacct

SEQ ID NO: 68 - DNA, 159 bp, Artificial Sequence
  Synthetic oligonucleotide
cccactagcugtattgtttaagaucggaagagcggtucagcaggaatgccgagaccgauctcgtatgccgtcutctgcttgaatgauacggcgaccaccgagauctacactctutccctacacgacgcucttccgatctatggucagcagcgggttaca

SEQ ID NO: 69 - DNA, 159 bp, Artificial Sequence
  Synthetic oligonucleotide
ctggcttttautgttagttaagaucggaagagcggtucagcaggaatgccgagaccgauctcgtatgccgtcutctgcttgaatgauacggcgaccaccgagauctacactctutccctacacgacgcucttccgatctacttttuccaacagagggaa

SEQ ID NO: 70 - DNA, 159 bp, Artificial Sequence
  Synthetic oligonucleotide
aaatgtcatcacautacttaagaucggaagagcggtucagcaggaatgccgagaccgauctcgtatgccgtcutctgcttgaatgauacggcgaccaccgagauctacactctutccctacacgacgcucttccgatctattaccaaaguttaccactt

SEQ ID NO: 71 - DNA, 159 bp, Artificial Sequence
  Synthetic oligonucleotide
tgtcccagaucgcattattaagaucggaagagcggtucagcaggaatgccgagaccgauctcgtatgccgtcutctgcttgaatgauacggcgaccaccgagauctacactctutccctacacgacgcucttccgatctagcaaugccatctttatcat

SEQ ID NO: 72 - DNA, 159 bp, Artificial Sequence
  Synthetic oligonucleotide
gctgtgtctacucatttgaaagaucggaagagcggtucagcaggaatgccgagaccgauctcgtatgccgtcutctgcttgaatgauacggcgaccaccgagauctacactctutccctacacgacgcucttccgatctaggcacccacaucatgtcat

SEQ ID NO: 73 - DNA, 159 bp, Artificial Sequence
  Synthetic oligonucleotide
accttauaagccagaatttaagaucggaagagcggtucagcaggaatgccgagaccgauctcgtatgccgtcutctgcttgaatgauacggcgaccaccgagauctacactctutccctacacgacgcucttccgatctaacaccutcacaatataccc

SEQ ID NO: 74 - DNA, 159 bp, Artificial Sequence
  Synthetic oligonucleotide
tctggaaacagucctgctccagaucggaagagcggtucagcaggaatgccgagaccgauctcgtatgccgtcutctgcttgaatgauacggcgaccaccgagauctacactctutccctacacgacgcucttccgatctaaatgaaauaaaatataaaa

SEQ ID NO: 75 - DNA, 159 bp, Artificial Sequence
  Synthetic oligonucleotide
gctgcguccccacgtcctgaagaucggaagagcggtucagcaggaatgccgagaccgauctcgtatgccgtcutctgcttgaatgauacggcgaccaccgagauctacactctutccctacacgacgcucttccgatctagatcacagacutcgggctg

SEQ ID NO: 76 - DNA, 159 bp, Artificial Sequence
  Synthetic oligonucleotide
ggctcgggccuctgcccccaagaucggaagagcggtucagcaggaatgccgagaccgauctcgtatgccgtcutctgcttgaatgauacggcgaccaccgagauctacactctutccctacacgacgcucttccgatctatgtgcccguccttgtccag

SEQ ID NO: 77 - DNA, 159 bp, Artificial Sequence
  Synthetic oligonucleotide
gagattcatuggaagcgaggagaucggaagagcggtucagcaggaatgccgagaccgauctcgtatgccgtcutctgcttgaatgauacggcgaccaccgagauctacactctutccctacacgacgcucttccgatctagagaagaaaacucaccttc

SEQ ID NO: 78 - DNA, 159 bp, Artificial Sequence
  Synthetic oligonucleotide
agttggatgtgguaggtaagagaucggaagagcggtucagcaggaatgccgagaccgauctcgtatgccgtcutctgcttgaatgauacggcgaccaccgagauctacactctutccctacacgacgcucttccgatctacgcgguagcagtagaagat

SEQ ID NO: 79 - DNA, 159 bp, Artificial Sequence
  Synthetic oligonucleotide
ccttgagccuggcctcacccagaucggaagagcggtucagcaggaatgccgagaccgauctcgtatgccgtcutctgcttgaatgauacggcgaccaccgagauctacactctutccctacacgacgcucttccgatcuaccggcagcagaagcugagt

SEQ ID NO: 80 - DNA, 159 bp, Artificial Sequence
  Synthetic oligonucleotide
ttttctgcaaaaucataactagaucggaagagcggtucagcaggaatgccgagaccgauctcgtatgccgtcutctgcttgaatgauacggcgaccaccgagauctacactctutccctacacgacgcucttccgatctaggactcugaagatgtacct

SEQ ID NO: 81 - DNA, 159 bp, Artificial Sequence
  Synthetic oligonucleotide
tatcaagactgugacttttaagaucggaagagcggtucagcaggaatgccgagaccgauctcgtatgccgtcutctgcttgaatgauacggcgaccaccgagauctacactctutccctacacgacgcucttccgatctaagcaagutgaggcactgaa

SEQ ID NO: 82 - DNA, 159 bp, Artificial Sequence
  Synthetic oligonucleotide
gctttgttatutgaagagcaagaucggaagagcggtucagcaggaatgccgagaccgauctcgtatgccgtcutctgcttgaatgauacggcgaccaccgagauctacactctutccctacacgacgcucttccgatctaaaaacatutttgtcttacc

SEQ ID NO: 83 - DNA, 159 bp, Artificial Sequence
  Synthetic oligonucleotide
tgggaagugctgcagctttaagaucggaagagcggtucagcaggaatgccgagaccgauctcgtatgccgtcutctgcttgaatgauacggcgaccaccgagauctacactctutccctacacgacgcucttccgatctaaatcucatagtttgacaat

SEQ ID NO: 84 - DNA, 159 bp, Artificial Sequence
  Synthetic oligonucleotide
ttgacaatauagacaatttaagaucggaagagcggtucagcaggaatgccgagaccgauctcgtatgccgtcutctgcttgaatgauacggcgaccaccgagauctacactctutccctacacgacgcucttccgatctaggaatcucatggcaaatag

SEQ ID NO: 85 - DNA, 159 bp, Artificial Sequence
  Synthetic oligonucleotide
taataggtcagacaautttaagaucggaagagcggtucagcaggaatgccgagaccgauctcgtatgccgtcutctgcttgaatgauacggcgaccaccgagauctacactctutccctacacgacgcucttccgatctagtcccaaggcauctcatcg

SEQ ID NO: 86 - DNA, 159 bp, Artificial Sequence
  Synthetic oligonucleotide
gatcttcaaaugatagtttaagaucggaagagcggtucagcaggaatgccgagaccgauctcgtatgccgtcutctgcttgaatgauacggcgaccaccgagauctacactctutccctacacgacgcucttccgatctatactggcaacaugactgtc

SEQ ID NO: 87 - DNA, 159 bp, Artificial Sequence
  Synthetic oligonucleotide
accaataaatuatagtcttaagaucggaagagcggtucagcaggaatgccgagaccgauctcgtatgccgtcutctgcttgaatgauacggcgaccaccgagauctacactctutccctacacgacgcucttccgatctaatagtgucagtagtagtga

SEQ ID NO: 88 - DNA, 159 bp, Artificial Sequence
  Synthetic oligonucleotide
ggttcagaaacaaaucgagtagaucggaagagcggtucagcaggaatgccgagaccgauctcgtatgccgtcutctgcttgaatgauacggcgaccaccgagauctacactctutccctacacgacgcucttccgatctaatatucagatgagcagttg

SEQ ID NO: 89 - DNA, 159 bp, Artificial Sequence
  Synthetic oligonucleotide
agcctattgatuatagtttaagaucggaagagcggtucagcaggaatgccgagaccgauctcgtatgccgtcutctgcttgaatgauacggcgaccaccgagauctacactctutccctacacgacgcucttccgatctatcaaaaugtaagccagtct

SEQ ID NO: 90 - DNA, 159 bp, Artificial Sequence
  Synthetic oligonucleotide
ttgcaaagttucttctattaagaucggaagagcggtucagcaggaatgccgagaccgauctcgtatgccgtcutctgcttgaatgauacggcgaccaccgagauctacactctutccctacacgacgcucttccgatctaaataugccacagatattcc

SEQ ID NO: 91 - DNA, 159 bp, Artificial Sequence
  Synthetic oligonucleotide
tacagaaagatguggaattaagaucggaagagcggtucagcaggaatgccgagaccgauctcgtatgccgtcutctgcttgaatgauacggcgaccaccgagauctacactctutccctacacgacgcucttccgatctaccaagaaacaauacagact

SEQ ID NO: 92 - DNA, 159 bp, Artificial Sequence
  Synthetic oligonucleotide
tattcttgcagaaugcattaagaucggaagagcggtucagcaggaatgccgagaccgauctcgtatgccgtcutctgcttgaatgauacggcgaccaccgagauctacactctutccctacacgacgcucttccgatctagaataaugcctccagttca

SEQ ID NO: 93 - DNA, 159 bp, Artificial Sequence
  Synthetic oligonucleotide
cagactcaaaaaauaatttaagaucggaagagcggtucagcaggaatgccgagaccgauctcgtatgccgtcutctgcttgaatgauacggcgaccaccgagauctacactctutccctacacgacgcucttccgatctattctgcuatgcccaaaggg

SEQ ID NO: 94 - DNA, 159 bp, Artificial Sequence
  Synthetic oligonucleotide
ccagggaaaaggcugaattaagaucggaagagcggtucagcaggaatgccgagaccgauctcgtatgccgtcutctgcttgaatgauacggcgaccaccgagauctacactctutccctacacgacgcucttccgatctaatgcugagagagttttctc

SEQ ID NO: 95 - DNA, 159 bp, Artificial Sequence
  Synthetic oligonucleotide
actttagcctctgautccttagaucggaagagcggtucagcaggaatgccgagaccgauctcgtatgccgtcutctgcttgaatgauacggcgaccaccgagauctacactctutccctacacgacgcucttccgatctaggggugggccttttttaga

SEQ ID NO: 96 - DNA, 159 bp, Artificial Sequence
  Synthetic oligonucleotide
cagtagtatuccaagaagtgagaucggaagagcggtucagcaggaatgccgagaccgauctcgtatgccgtcutctgcttgaatgauacggcgaccaccgagauctacactctutccctacacgacgcucttccgatctagtagaccuatacagtctcc

SEQ ID NO: 97 - DNA, 159 bp, Artificial Sequence
  Synthetic oligonucleotide
aagcuccaagcccaaccttaagaucggaagagcggtucagcaggaatgccgagaccgauctcgtatgccgtcutctgcttgaatgauacggcgaccaccgagauctacactctutccctacacgacgcucttccgatctaatcagaugaataatggtaa

SEQ ID NO: 98 - DNA, 159 bp, Artificial Sequence
  Synthetic oligonucleotide
tgccagagtgacuccttttaagaucggaagagcggtucagcaggaatgccgagaccgauctcgtatgccgtcutctgcttgaatgauacggcgaccaccgagauctacactctutccctacacgacgcucttccgatctaagatucaaaagataatcag

SEQ ID NO: 99 - DNA, 159 bp, Artificial Sequence
  Synthetic oligonucleotide
taccttgtgacauctgtttaagaucggaagagcggtucagcaggaatgccgagaccgauctcgtatgccgtcutctgcttgaatgauacggcgaccaccgagauctacactctutccctacacgacgcucttccgatctatuacaacccaagccctagg

SEQ ID NO: 100 - DNA, 159 bp, Artificial Sequence
  Synthetic oligonucleotide
acacatggcaugacgtgactagaucggaagagcggtucagcaggaatgccgagaccgauctcgtatgccgtcutctgcttgaatgauacggcgaccaccgagauctacactctutccctacacgacgcucttccgatctaggaacgugggcatgacctg

SEQ ID NO: 101 - DNA, 159 bp, Artificial Sequence
  Synthetic oligonucleotide
agataggguggaaaagaaacagaucggaagagcggtucagcaggaatgccgagaccgauctcgtatgccgtcutctgcttgaatgauacggcgaccaccgagauctacactctutccctacacgacgcucttccgatctatttugaaactgaaagatcc

SEQ ID NO: 102 - DNA, 159 bp, Artificial Sequence
  Synthetic oligonucleotide
ggcagagagaaaacagaauaagaucggaagagcggtucagcaggaatgccgagaccgauctcgtatgccgtcutctgcttgaatgauacggcgaccaccgagauctacactctutccctacacgacgcucttccgatctactccccuagattccttctg

SEQ ID NO: 103 - DNA, 159 bp, Artificial Sequence
  Synthetic oligonucleotide
gggatgtgtgggcuaaatgtagaucggaagagcggtucagcaggaatgccgagaccgauctcgtatgccgtcutctgcttgaatgauacggcgaccaccgagauctacactctutccctacacgacgcucttccgatctaaggaaacutctctctaaag

SEQ ID NO: 104 - DNA, 159 bp, Artificial Sequence
  Synthetic oligonucleotide
gtcacattaugcaagacactagaucggaagagcggtucagcaggaatgccgagaccgauctcgtatgccgtcutctgcttgaatgauacggcgaccaccgagauctacactctutccctacacgacgcucttccgatctacatgttutagttcattttt

SEQ ID NO: 105 - DNA, 159 bp, Artificial Sequence
  Synthetic oligonucleotide
ataaagggaaaaggauctcaagaucggaagagcggtucagcaggaatgccgagaccgauctcgtatgccgtcutctgcttgaatgauacggcgaccaccgagauctacactctutccctacacgacgcucttccgatctaaatgtgauagtgtctgtgt

SEQ ID NO: 106 - DNA, 159 bp, Artificial Sequence
  Synthetic oligonucleotide
aacatgatauacaaaaatttagaucggaagagcggtucagcaggaatgccgagaccgauctcgtatgccgtcutctgcttgaatgauacggcgaccaccgagauctacactctutccctacacgacgcucttccgatctatttacuaaagagattagtg

SEQ ID NO: 107 - DNA, 159 bp, Artificial Sequence
  Synthetic oligonucleotide
tctgaacgggguccggtttaagaucggaagagcggtucagcaggaatgccgagaccgauctcgtatgccgtcutctgcttgaatgauacggcgaccaccgagauctacactctutccctacacgacgcucttccgatctaaaauaaacctgcctctatg

SEQ ID NO: 108 - DNA, 119 bp, Artificial Sequence
  Synthetic oligonucleotide
agatcggaagagcgtcgtgtagggaaagagtgtagatctcggtggtcgccgtatcattcaagcagaagacggcatacgagatcggtctcggcattcctgctgaaccgctcttccgatct